(19) United States
(12) Patent Application Publication
(10) Pub. No.: US 2015/0056231 A1
(43) Pub. Date: Feb. 26, 2015

(54) RECOMBINANT PAPAYA MOSAIC VIRUS COAT PROTEINS AND USES THEREOF IN INFLUENZA VACCINES

(71) Applicant: FOLIA BIOTECH INC., Quebec City (CA)

(72) Inventors: Denis Leclerc, Fossambault-Su- Le Lac (CA); Nathalie Majeau, Fossambault-Su- Le Lac (CA)

(21) Appl. No.: 14/388,810

(22) PCT Filed: Feb. 19, 2013
(86) PCT No.: PCT/CA2013/050127

§ 371 Date: Sep. 28, 2014

Related U.S. Application Data
(60) Provisional application No. 61/618,972, filed on Apr. 2, 2012.
(60) Provisional application No. 61/694,422, filed on Aug. 29, 2012.

(51) Int. Cl. C07K 14/005; A61K 39/12; C12N 7/00; A61K 39/145

(52) U.S. Cl. CPC C07K 14/005; A61K 39/145; A61K 39/12; C12N 7/00; A61K 2039/5258
USPC 424/186.1; 530/350; 514/21.2

(57) ABSTRACT

Recombinant papaya mosaic virus (PapMV) coat proteins comprising one or more antigenic peptides derived from an influenza virus antigen, such as from the M2e peptide, fused at a position within a predicted random coil within 13 amino acids of the N-terminus of the coat protein, uses thereof to prepare virus-like particles (VLPs), and uses of the VLPs in influenza vaccines.

### FIELD OF THE INVENTION

The present invention relates to the field of immunogenic formulations and, in particular, to formulations comprising recombinant papaya mosaic virus coat proteins for use to induce an immune response against an influenza virus.

### BACKGROUND OF THE INVENTION

The ability of papaya mosaic virus (PapMV) VLPs to act as immunopotentiators and adjuvants has been described in the following patent and patent applications.

U.S. Pat. No. 7,641,896, Canadian Patent Application No. 2,434,000, and International Patent Application No. PCT/CA03/00985 (WO 2004/004761) describe the use of PapMV or VLPs derived from PapMV coat protein for potentiating immune responses in an animal. Also described are fusions of PapMV coat proteins with immunogens.

International Patent Application No. PCT/CA2007/002069 (WO 2008/058396) describes influenza vaccines based on PapMV and PapMV VLPs. The vaccines comprise PapMV or PapMV VLPs combined with, or fused to, one or more influenza antigens.

International Patent Application No. PCT/CA2007/001904 (WO 2008/058369) describes immunogenic affinity-conjugated antigen systems based on PapMV. Fusions of PapMV coat protein with a plurality of affinity peptides capable of binding an antigen of interest are described in which the affinity peptides are attached to the coat protein by chemical means or by genetic fusion.

International Patent Application No. PCT/CA2008/000154 (WO 2008/089569) describes vaccines against S. typhi and other enterobacterial pathogens based on PapMV. Fusion of PapMV coat proteins with one or more enterobacterial antigens is described.

International Patent Application No. PCT/CA2009/00636 (WO 2010/012069) describes multivalent vaccines based on PapMV, including combination of PapMV or VLPs with commercial influenza vaccines.

Other publications have described the ability of PapMV VLPs to elicit humoral and cellular immune responses (Denis et al., 2007, Virology, 363:59-68; Denis et al., 2008, Vaccine, 26:3395-3403; Leclerc et al., 2007, J Virol., 81:1319-26, and Lacasse et al., 2008, J. Virol., 2008; 82:785-94).

Mutations at the N-terminus of the PapMV coat protein and their effect on PapMV-host interactions have been described (Ikegami, “Papaya Mosaic Potexvirus as an Expression Vector for Foreign Peptides,” M.Sc. Thesis, 1995, National Library of Canada, Ottawa). The mutations included a conservative lysine to arginine substitution at position 3 of the wild-type sequence, an 18 amino acid deletion downstream of position 3, and an 11 amino acid in-frame insertion as an addition to the N-terminus and a replacement of the wild-type N-terminal sequence. All three mutants were able to produce local lesions in C. globosa and to infect the systemic host C. papaya, suggesting that the mutants were able to assemble and move systemically.

Fusion of the HA11 peptide to several putative surface-exposed sites in the PapMV coat protein has also been investigated (Rioux et al., 2012, PLoS ONE, 7(2), e31925). Fusion of the peptide at positions 12 and 187 of the coat protein resulted in fusion proteins capable of self-assembly into VLPs. VLPs comprising fusion of the peptide at position 12 of the coat protein were stable and able to induce an immune response to the HA11 peptide.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide recombinant papaya mosaic virus coat proteins and uses thereof to induce an immune response in a subject against an influenza virus. In accordance with one aspect of the invention, there is provided a fusion protein comprising a peptide antigen derived from influenza M2e peptide fused to a papaya mosaic virus (PapMV) coat protein after an amino acid that corresponds to any one of amino acids 6 to 12 of SEQ ID NO:1, wherein the fusion protein is capable of self-assembly to form a virus-like particle (VLP), and wherein the peptide antigen is 20 amino acids or less in length and comprises the general sequence: V-X1-T-X2-X3-X4-X5 [SEQ ID NO:96], wherein X1 is E or D; X2 is P or L; X3 is T or I; X4 is R or K, and X5 is N, S or K.

In accordance with another aspect of the invention, there is provided a virus-like particle (VLP) comprising the fusion protein.

In accordance with another aspect of the invention, there is provided a pharmaceutical composition comprising the VLP and a pharmaceutically acceptable carrier.

In accordance with another aspect of the invention, there is provided a method of inducing an immune response against an influenza virus in a subject comprising administering to the subject an effective amount of the VLP.

In accordance with another aspect of the invention, there is provided a method of reducing the risk of a subject developing influenza comprising administering to the subject an effective amount of the VLP.

In accordance with another aspect of the invention, there is provided a method of immunizing a subject against infection with an influenza virus comprising administering to the subject an effective amount of the VLP.

In accordance with another aspect of the invention, there is provided a virus-like particle (VLP) comprising the above fusion protein for use to induce an immune response against an influenza virus in a subject in need thereof.

In accordance with another aspect of the invention, there is provided a use of a virus-like particle (VLP) comprising the fusion protein to induce an immune response against an influenza virus in a subject in need thereof.

In accordance with another aspect of the invention, there is provided a use of a virus-like particle (VLP) comprising the fusion protein in the manufacture of a medicament for inducing an immune response against an influenza virus in a subject.

In accordance with another aspect of the invention, there is provided a virus-like particle (VLP) comprising the fusion protein for use to reduce the risk of a subject developing influenza.

In accordance with another aspect of the invention, there is provided a use of a virus-like particle (VLP) comprising the fusion protein to reduce the risk of a subject developing influenza.

In accordance with another aspect of the invention, there is provided a use of a virus-like particle (VLP) comprising the fusion protein in the manufacture of a medicament for reducing the risk of a subject developing influenza.

In accordance with another aspect of the invention, there is provided a virus-like particle (VLP) comprising the fusion protein for use to immunize a subject against infection with an influenza virus.

In accordance with another aspect of the invention, there is provided a use of a virus-like particle (VLP) comprising the fusion protein to immunize a subject against infection with an influenza virus.

In accordance with another aspect of the invention, there is provided a use of a virus-like particle (VLP) comprising the fusion protein in the manufacture of a medicament for immunizing a subject against infection with an influenza virus.

In accordance with another aspect of the invention, there is provided a pharmaceutical kit comprising the above VLP and instructions for use.

In accordance with another aspect of the invention, there is provided a fusion protein comprising one or more peptide antigens fused to a papaya mosaic virus (PapMV) coat protein after an amino acid that corresponds to any one of amino acids 6 to 12, 185 to 192 and 197 to 214 of SEQ ID NO:1, wherein the fusion protein is capable of self-assembly to form a virus-like particle (VLP), and wherein the VLP is stable at a temperature of at least 25° C.

In accordance with another aspect of the invention, there is provided a method of identifying a virus-like particle (VLP) fused to a peptide antigen that is capable of potentiating an immune response to the peptide antigen in a subject, the method comprising the steps of: providing a VLP comprising Papaya mosaic virus (PapMV) coat protein fused to the peptide antigen, and determining the stability of the VLP at a temperature of at least 25° C., wherein stability at a temperature of at least 25° C. is indicative of a VLP capable of potentiating an immune response to the peptide antigen.

### BRIEF DESCRIPTION OF THE DRAWINGS

These and other features of the invention will become more apparent in the following detailed description in which reference is made to the appended drawings.

FIG. 1 presents (A) the amino acid sequence of the wild-type PapMV coat protein (SEQ ID NO:1); (B) the nucleotide sequence of the wild-type PapMV coat protein (SEQ ID NO:2); (C) the amino acid sequence of the modified PapMV coat protein CPΔN5 (SEQ ID NO:4), and (D) the amino acid sequence of modified PapMV coat protein PapMV CPsm (SEQ ID NO:5).

FIG. 2 presents the nucleic acid and amino acid sequences of the recombinant PapMV coat proteins described in the Examples section with inserted sequences corresponding to the antigenic peptide marked in bold and underlined: (A) nucleic acid and amino acid sequence of PapMV NP-8 [SEQ ID NOs:72 and 73, respectively]; (B) nucleic acid and amino acid sequence of PapMV NP-183 [SEQ ID NOs:74 and 75, respectively]; (C) nucleic acid and amino acid sequence of PapMV NP-C [SEQ ID NOs:76 and 77, respectively]; (D) nucleic acid and amino acid sequence of PapMV Loop6-8 [SEQ ID NOs:78 and 79, respectively]; (E) nucleic acid and amino acid sequence of PapMV Loop6-183 [SEQ ID NOs:80 and 81, respectively]; (F) nucleic acid and amino acid sequence of PapMV Loop6-C [SEQ ID NOs:82 and 83, respectively]; (G) nucleic acid and amino acid sequence of PapMV 3NP-C [SEQ ID NOs:84 and 85, respectively]; (H) nucleic acid and amino acid sequence of PapMV NP-8/183 [SEQ ID NOs:86 and 87, respectively]; (I) nucleic acid and amino acid sequence of PapMV NP-8/C [SEQ ID NOs:88 and 89, respectively]; (J) nucleic acid and amino acid sequence of PapMV NP-183/C [SEQ ID NOs:90 and 91, respectively]; (K) nucleic acid and amino acid sequence of PapMV 3NP-8 [SEQ ID NOs:92 and 93, respectively], and (L) PapMV 3NP-8/183/C (PapMV triple NP) [SEQ ID NOs:6 and 94, respectively].

FIG. 3 presents the amino acid sequences for the PapMV coat protein-M2e peptide fusions described in Example 1: (A) Construct #1 [SEQ ID NO:23]; (B) Construct #2 [SEQ ID NO:24]; (C) Construct #3 [SEQ ID NO:25]; (D) Construct #4 [SEQ ID NO:26]; (E) Construct #5 [SEQ ID NO:27] and (F) Construct #6 [SEQ ID NO:28]. The inserted M2e peptide sequences are shown in bold.

FIG. 4 presents the secondary structure prediction of the PapMV coat protein (CP) (taken from Lecours et al., 2006, PEP, 47:273-80) showing the locations in the PapMV CP amino acid sequence (SEQ ID NO:4) at which the HA11 peptide sequences were inserted in Rioux et al. (2012, PLoS ONE, 7(2), e31925).

FIG. 5 illustrates the positions and sequences of the M2e peptide fusions to the PapMV coat protein for the constructs tested in Example 1 (inserted sequences are shown in bold and underlined).

FIG. 6 illustrates the denaturation of the PapMV-M2e constructs of Example 1 observed by binding of Sypro-Orange to hydrophobic residues.

FIG. 7 shows transmission electron micrographs of the thermostable constructs (#1, 2, 3, 4, 5, 6, 9 and 10) from FIG. 5.

FIG. 8 presents the results of an evaluation of the immunogenicity of the VLPs comprising PapMV-M2e constructs of Example 1. BALB/c mice, 5 per group, were immunized twice (on days 1 and 14) with the VLPs. Blood samples were obtained 14 days after each immunization and the humoral response measured by ELISA. A) Total anti-M2e IgG titers at days 14 and 28, B) anti-M2e IgG2a titers at days 14 and 28. *** p<0.001, ** p<0.01, * p<0.1.

FIG. 9 presents (A) the amino acid sequence of PapMV CP at the site of fusion of the S. typhi loop 6 peptide; (B) SDS-PAGE analysis of the production of recombinant PapMV CP fusion proteins: bacterial lysate before induction (Lane 1) and after expression (Lane 2) of the protein PapMV-loop-6-8; Lane 3: purified PapMV-loop-6-8; bacterial lysate before induction (Lane 4) and after expression (Lane 5) of the protein PapMV-loop-6-183; Lane 6: purified PapMV-loop-6-183; bacterial lysate before induction (Lane 7) and after expression (Lane 8) of the protein PapMV-loop-6-C; and Lane 9: purified PapMV-loop-6-C; (C) electron micrographs of VLPs comprising PapMV loop-6-8, PapMV-loop-6-183 and PapMV-loop-6-C, and (D) dynamic light scattering (DLS) of VLPs comprising PapMV loop-6-8, PapMV-loop-6-183 and PapMV-loop-6-C.

FIG. 10 presents data depicting the humoral response to PapMV SM (or WT), PapMV loop-6-8 (loop6-8), PapMV-loop-6-183 (loop6-183) and PapMV-loop-6-C (loop6-C): total IgG (A) and the IgG2a (B) directed to the PapMV platform and the total IgG (C) and IgG2a (D) directed to loop-6 peptide were measured by ELISA.

FIG. 11 presents (A) the amino acid sequence of PapMV CP at the site of fusion with the NP peptide; (B) SDS-PAGE showing expression of PapMV proteins and VLPs fused to the NP CTL epitope; (C) Electron micrograph of VLPs comprising PapMV NP-8, PapMV NP-183 and PapMV NP-C, and (D) Dynamic light scattering (DLS) of the PapMV NP-8, PapMV NP-183 and PapMV NP-C VLPs and discs.

FIG. 12 presents the results of an ELISPOT analysis showing IFN-γ secretion in mice after vaccination with PapMV VLPs and discs comprising PapMV CP fused to the influenza NP₁₄₇₋₁₅₅ peptide, (A) VLPs comprising PapMV NP-12, PapMV NP-187, PapMV NP-C or PapMV CP (***p≦0.001 compared to all groups); (B) VLPs and discs comprising PapMV NP-12 or PapMV CP (***p≦0.001 compared to all groups), and (C) VLPs comprising PapMV NP-12, PapMV NP-C or PapMV CP cross-linked with glutaraldehyde (Glut) or not cross-linked (*p≦0.05 and **p≦0.01).

FIG. 13 presents (A) the amino acid sequence of PapMV CP at the sites of the fusions with the NP₁₄₇₋₁₅₅ peptide; (B) SDS-PAGE showing expression of PapMV proteins and VLPs harbouring multiple copies of the NP₁₄₇₋₁₅₅ peptide, and (C) Dynamic light scattering (DLS) of the PapMV VLPs 3NP-C, NP-8/183, NP-8/C, NP-8/C and triple NP.

FIG. 14 depicts the results from a microarray analysis of 27 overlapping peptides from the PapMV CP hybridized with the serum of mice immunized with PapMV VLPs; the threshold was positioned at the relative fluorescence intensity of peptide 1, as it is known to be surface-exposed.

FIG. 15 presents the results of electron microscopy and dynamic light scattering analysis of chemically modified PapMV VLPs and shows that VLPs treated with DEPC or EDC did not sustain disruption of their quaternary structure, as shown both by electron microscopy (A) and dynamic light scattering (B).

FIG. 16 presents the amino acid sequence of the wild-type PapMV coat protein [SEQ ID NO:1] on which the amino acid residues involved in the predicted random coils at the C- and N-termini are marked in bold and underlined.

FIG. 17 presents the results of an ELISPOT analysis of PapMV VLPs and discs comprising PapMV CP fused to multiple copies the influenza NP₁₄₇₋₁₅₅ peptide.

FIG. 18 presents charts depicting changes in structure of VLPs comprising PapMV CP fused to the influenza NP₁₄₇₋₁₅₅ peptide as measured by dynamic light scattering (A) VLPs comprising recombinant PapMV CP NP₁₄₇₋₁₅₅ peptide fusions compared to PapMV VLPs without fusion showing the aggregation of PapMV NP-187 and NP-C VLPs at temperatures below mice body temperature, and (B) cross-linked PapMV NP-C VLPs showing a higher temperature stability; and (C) results from trypsin digests of VLPs comprising recombinant PapMV CP NP₁₄₇₋₁₅₅ peptide with or without cross-linking by glutaraldehyde.

FIG. 19 depicts the MS/MS spectra of digested peptides containing chemical modifications by EDC: regions that contain modifications are from V16 to K30 (A), from M122 to K137 (B) and from G199 to R221 (C). The underlined product ions contain the EDC modification.

FIG. 20 depicts the MS/MS spectra of digested peptides containing chemical modifications by DEPC: regions that contain modifications are from M122 to K137 (A) and from G199 to R221 (B). The DEPC modification in B cannot be located precisely and is therefore at either one of the two threonines. The underlined product ions contain the DEPC modification.

### DETAILED DESCRIPTION OF THE INVENTION

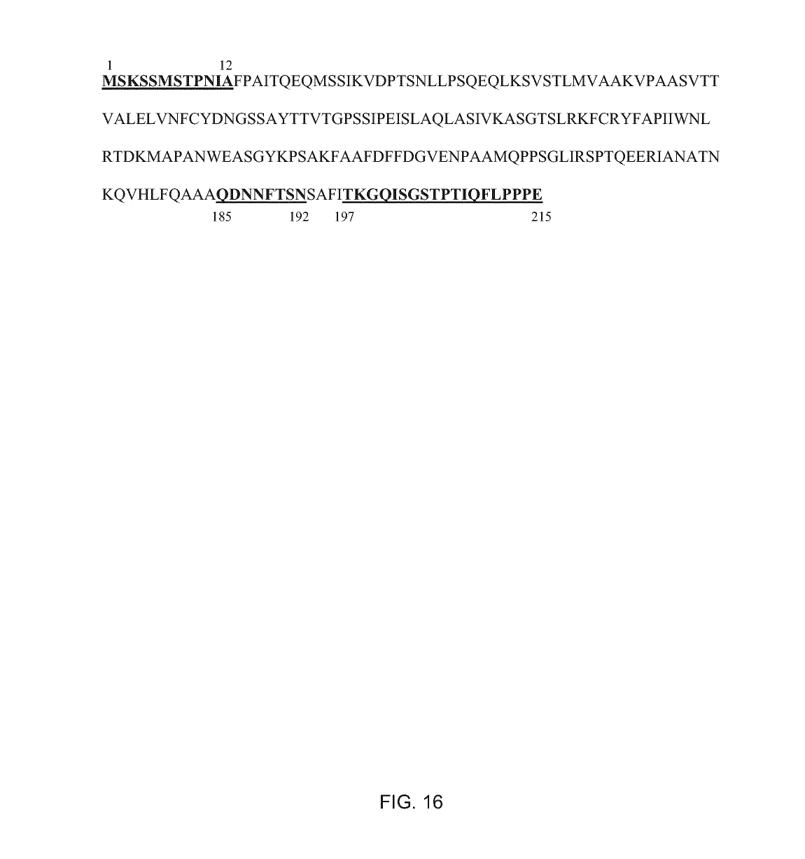

The present invention relates to recombinant PapMV coat proteins comprising one or more antigenic peptides fused within a coat protein (CP) “surface-coil” region, specifically, within a predicted random coil comprising 13 amino acids of the N-terminus of the wild-type CP (SEQ ID NO:1; see FIG. 16). The recombinant PapMV CPs comprising the fused antigenic peptide(s) are capable of self-assembly to form virus-like particles (VLPs).

In certain embodiments, the one or more antigens are derived from the influenza virus, preferably from the M2e peptide, and are inserted into the PapMV CP after any one of amino acids 6-12 of SEQ ID NO:1, or positions corresponding thereto, for example, after any one of amino acids 1-8 of SEQ ID NO:4. Virus-like particles (VLPs) prepared from these recombinant coat proteins are useful to induce a protective immune response against the influenza virus. Some embodiments, therefore, relate to the use of these VLPs to induce a protective immune response against an influenza virus in a mammal, such as a human. In certain embodiments, it is contemplated that the VLPs may be used as influenza vaccines.

Certain embodiments of the invention relate to recombinant PapMV CPs comprising a fusion of an antigenic peptide derived from an influenza virus, such as from the M2e peptide, after a position corresponding to any one of amino acids 6, 7 or 10 of the PapMV CP sequence shown in SEQ ID NO:1, for example, after amino acids 2, 3 or 6 of the PapMV CP sequence shown in SEQ ID NO:4. In some embodiments, the antigenic peptide is an M2e-derived peptide comprising the general sequence: V-X1-T-X2-X3-X4-X5 [SEQ ID NO:96], where X1 is E or D; X2 is P or L; X3 is T or I; X4 is R or K, and X5 is N, S or K. As demonstrated herein, VLPs comprising PapMV CP fused to an M2e-derived peptide comprising the general sequence of SEQ ID NO:96 after the position corresponding to any one of amino acids 6, 7 or 10 of the PapMV CP sequence shown in SEQ ID NO:1 are capable of providing a protective immune response against influenza virus with a single immunization.

Certain embodiments contemplate that the recombinant PapMV CP may further comprise one or more antigenic peptides fused at a second surface coil region and/or at the C-terminus of the CP.

As described herein, in some embodiments, the ability of the VLPs comprising the recombinant CP to trigger an effective immune response to the fused peptide can be predicted based on the thermostability of the VLP. Thus, in certain embodiments, VLPs comprising the recombinant CP are selected to be stable at a temperature of at least 30° C.

### DEFINITIONS

Unless defined otherwise, all technical and scientific terms used herein have the same meaning as commonly understood by one of ordinary skill in the art to which this invention belongs.

As used herein, the term “about” refers to approximately a +/−10% variation from a given value. It is to be understood that such a variation is always included in any given value provided herein, whether or not it is specifically referred to.

The term “immunogenic,” as used herein, refers to the ability of a substance to induce a detectable immune response in an animal.

The term “immune response,” as used herein, refers to an alteration in the reactivity of the immune system of an animal in response to administration of a substance (for example, a compound, molecule, material or the like) and may involve antibody production, induction of cell-mediated immunity, complement activation, development of immunological tolerance, or a combination thereof.

The term “vaccination,” as used herein, refers to the administration of a vaccine to a subject for the purposes of generating a beneficial immune response. Vaccination may have a prophylactic effect, a therapeutic effect, or a combination thereof. Vaccination can be accomplished using various methods depending on the subject to be treated including, but not limited to, parenteral administration, such as intraperitoneal injection (i.p.), intravenous injection (i.v.) or intramuscular injection (i.m.); oral administration; intranasal administration; intradermal administration; transdermal administration and immersion.

The term “vaccine,” as used herein, refers to a composition capable of producing a beneficial immune response.

“Naturally-occurring,” as used herein, as applied to an object, refers to the fact that the object can be found in nature. For example, an organism (including a virus), or a polypeptide or polynucleotide sequence that is present in an organism that can be isolated from a source in nature and which has not been intentionally modified by man in the laboratory is naturally-occurring.

The terms “polypeptide” or “peptide” as used herein is intended to mean a molecule in which there is at least two amino acids, for example at least four amino acids, linked by peptide bonds.

The term “virus-like particle” (VLP), as used herein, refers to a self-assembling particle which has a similar physical appearance to a virus particle. The VLP may or may not comprise nucleic acids. VLPs are generally incapable of replication.

The term “disc,” as used herein, refers to a multimeric form of a PapMV coat protein that comprises about 18 to about 22 subunits and has a molecular weight of about 400 kDa to about 500 kDa) (Tremblay et al., 2006, FEBS, 273:14-25). In contrast to a non-specific aggregate that does not have a defined structure, a disc appears as a substantially spherical structure having a diameter of about 40 nm or less, as measured by DLS.

The term “antigen” as used herein refers to a molecule, molecules, a portion or portions of a molecule, or a combination of molecules, up to and including whole cells and tissues, which are capable of inducing an immune response in a subject alone or in combination with an adjuvant. The immunogen/antigen may comprise a single epitope or may comprise a plurality of epitopes. The term thus encompasses peptides, carbohydrates, proteins, nucleic acids, and various microorganisms, in whole or in part, including viruses, bacteria and parasites. Haptens are also considered to be encompassed by the term “antigen” as used herein.

The term “subject” or “patient” as used herein refers to an animal in need of vaccination and/or treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep or other hoofed animals, dogs, cats, chickens, ducks, non-human primates, guinea pigs, rabbits, ferrets, rats, hamsters and mice.

The term “substantially identical,” as used herein in relation to a nucleic acid or amino acid sequence indicates that, when optimally aligned, for example using the methods described below, the nucleic acid or amino acid sequence shares at least 70%, at least 75%, at least 80%, at least 85%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The term “plurality” as used herein means more than one, for example, two or more, three or more, four or more, and the like.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method, use or composition of the invention, and vice versa. Furthermore, compositions and kits of the invention can be used to achieve methods and uses of the invention.

### Recombinant PapMV Coat Proteins

The recombinant PapMV coat proteins (CPs) according to the present invention comprise one or more antigenic peptides derived from an antigen fused to a PapMV CP at a position within a CP N-terminal surface-coil region.

In some embodiments, a recombinant CP may comprise a plurality of antigenic peptides (i.e. two or more), and the plurality of peptides may be fused within the N-terminal surface-coil region or they may each be fused within a different surface-coil region and/or at the C-terminus.

### PapMV Coat Protein

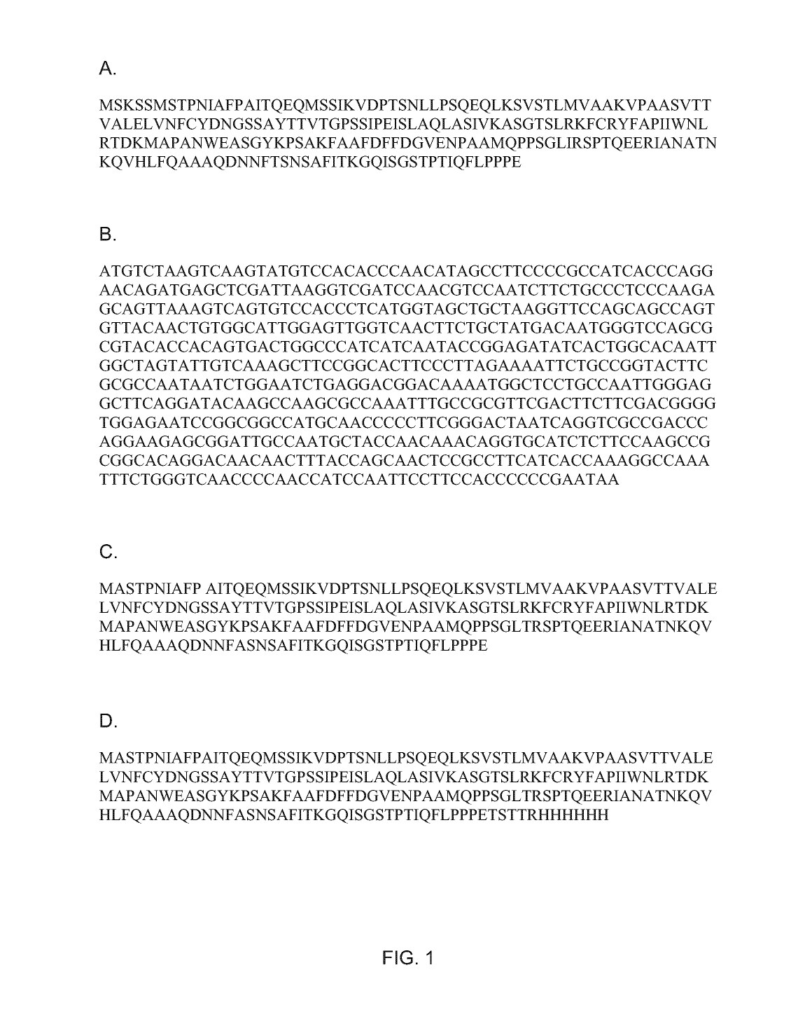

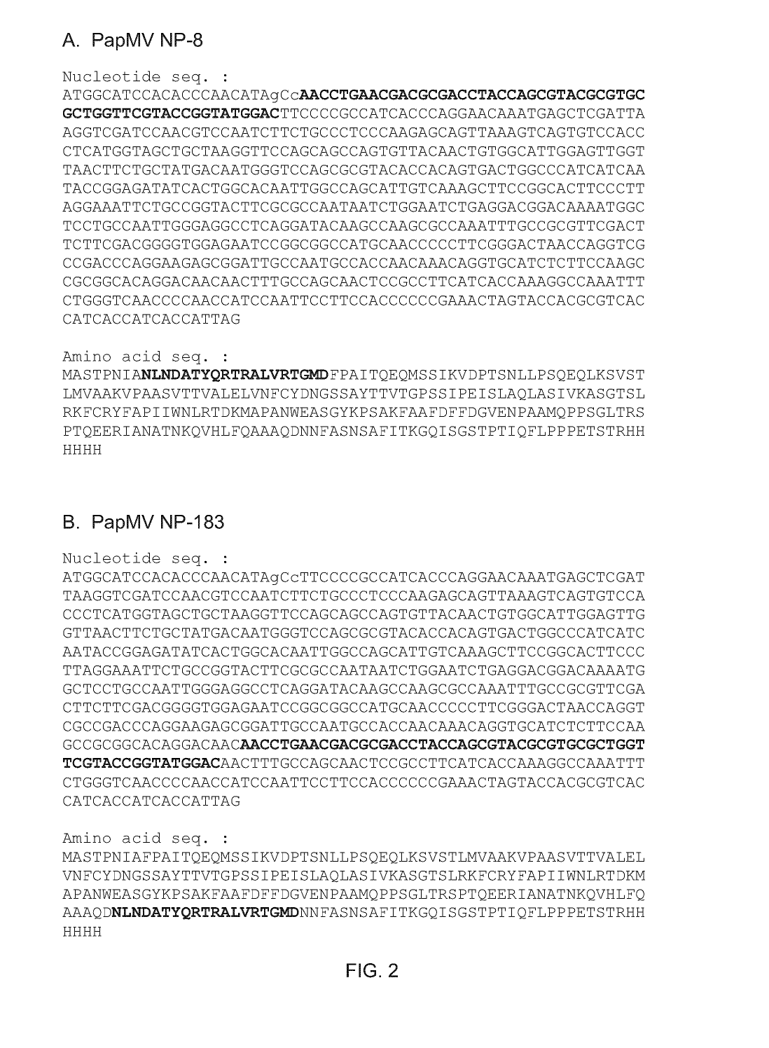

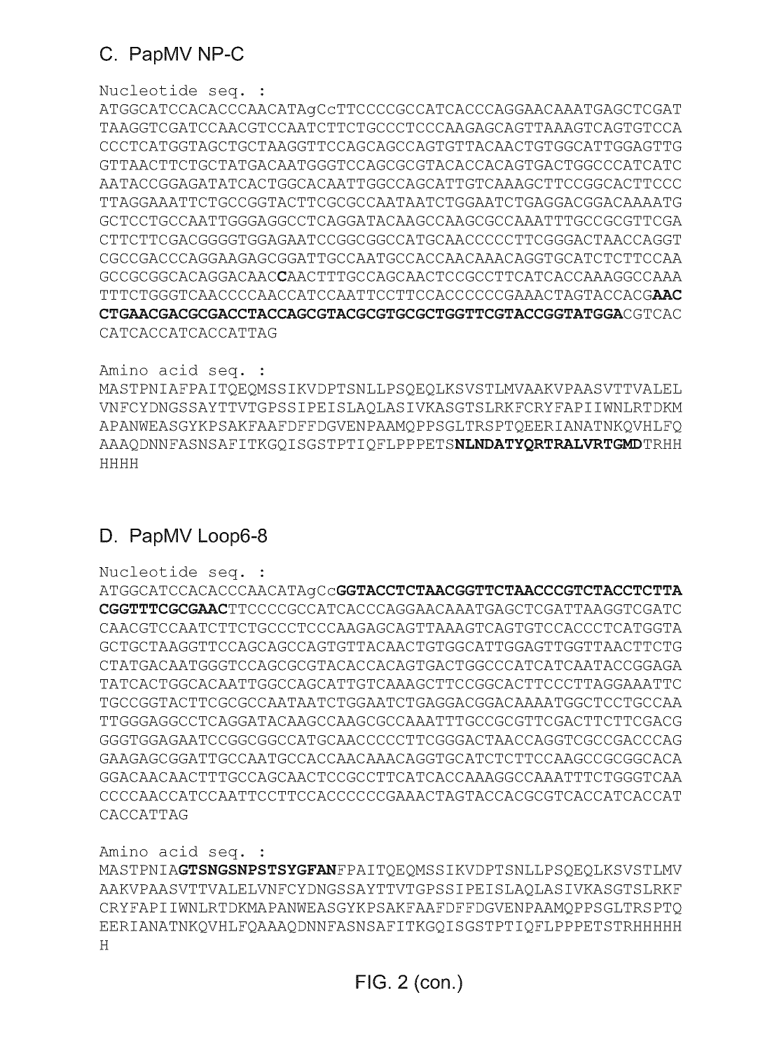

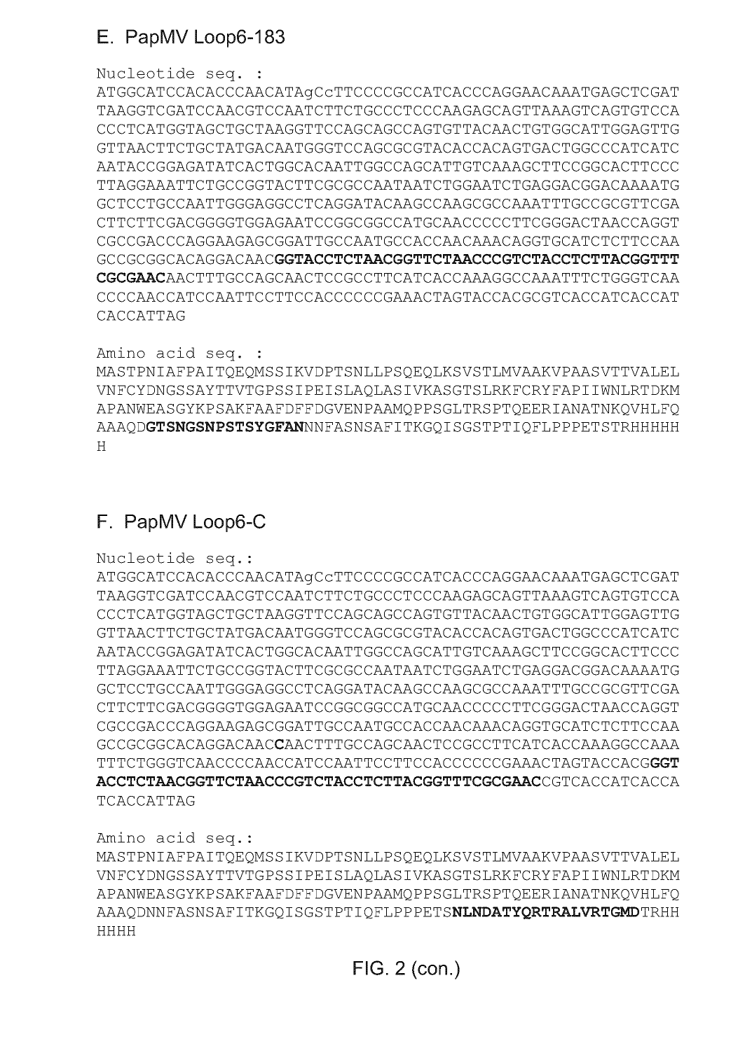

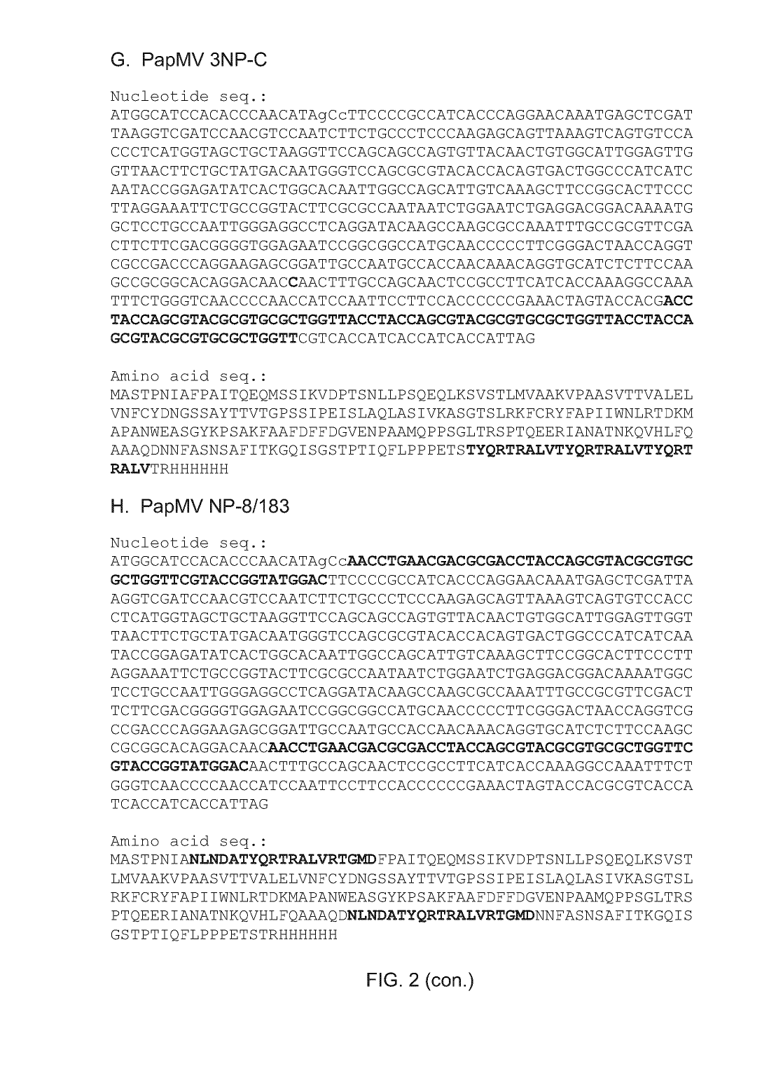

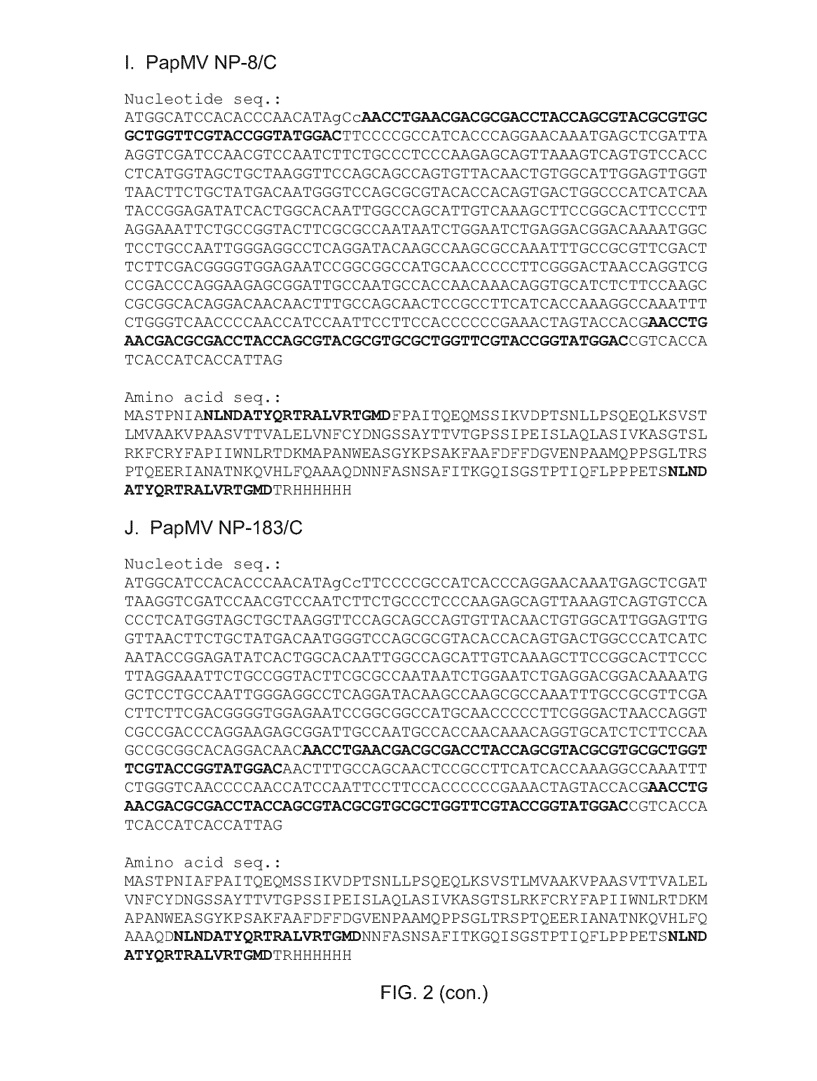

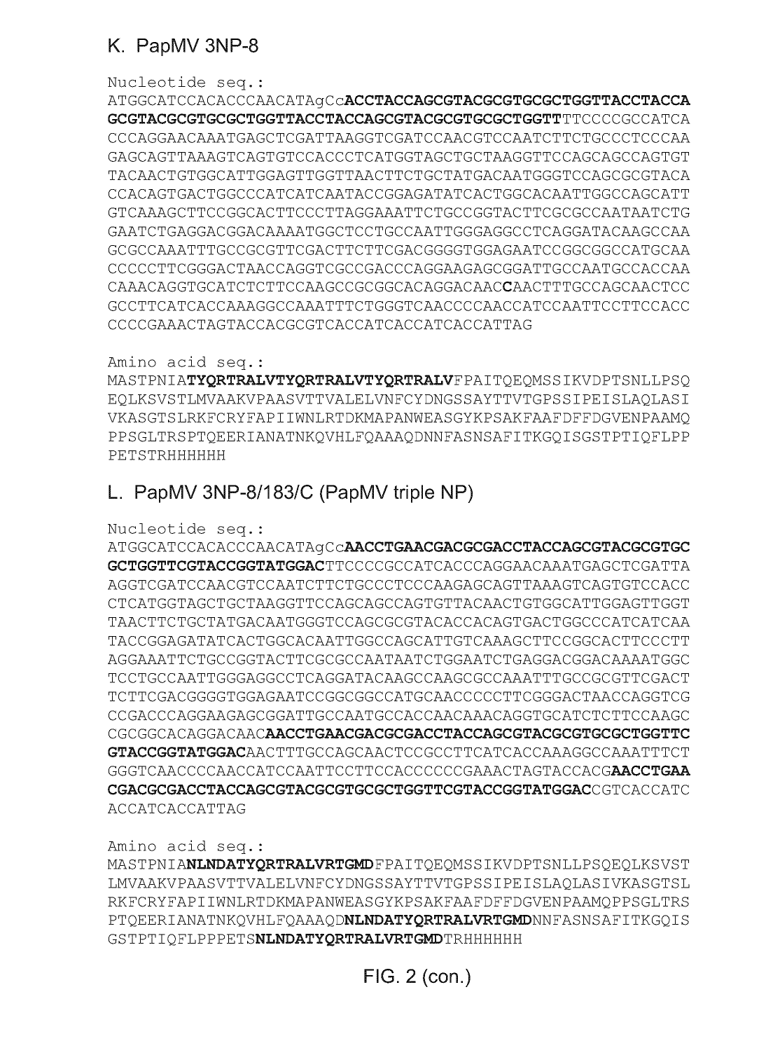

The PapMV coat protein used to prepare the recombinant PapMV CPs according to the invention can be the entire PapMV CP, or part thereof, or it can be a genetically modified version of the wild-type PapMV CP, for example, comprising one or more amino acid deletions, insertions, replacements and the like, provided that the CP retains the ability to self-assemble into VLPs. The amino acid sequence of the wild-type PapMV coat (or capsid) protein is known in the art (see, Sit, et al., 1989, J. Gen. Virol., 70:2325-2331, and GenBank Accession No. NP_{—}044334.1) and is provided herein as SEQ ID NO:1 (see FIG. 1A). The nucleotide sequence of the PapMV CP is also known in the art (see, Sit, et al., ibid., and GenBank Accession No. NC_{—}001748 (nucleotides 5889-6536)) and is provided herein as SEQ ID NO:2 (see FIG. 1B).

As noted above, the amino acid sequence of the PapMV CP used to prepare the recombinant PapMV CPs need not correspond precisely to the parental (wild-type) sequence, i.e. it may be a “variant sequence.” For example, the PapMV CP may be mutagenized by substitution, insertion or deletion of one or more amino acid residues so that the residue at that site does not correspond to the parental (reference) sequence. One skilled in the art will appreciate, however, that such mutations will not be extensive and will not dramatically affect the ability of the recombinant PapMV CP to self-assemble into VLPs. The ability of a variant version of the PapMV CP to self-assemble into VLPs can be assessed, for example, by electron microscopy following standard techniques, such as the exemplary methods set out in the Examples provided herein.

Naturally occurring variants of PapMV CP are also known. For example, Noa-Carrazana and Silva-Rosales (Plant Disease, 2001, 85:558) reported the identification of two Mexican isolates of PapMV which had coat proteins that shared a sequence similarity of 88% with the PapMV coat protein sequence deposited under GenBank Accession No. D13957 (i.e. SEQ ID NO:1) and a sequence similarity with each other of 94%. Such naturally occurring variants are also contemplated in certain embodiments of the invention.

Also contemplated in some embodiments are recombinant PapMV CPs prepared using fragments of the wild-type CP that retain the ability to self-assemble into a VLP (i.e. are “functional” fragments). For example, a fragment may comprise a deletion of one or more amino acids from the N-terminus, the C-terminus, or the interior of the protein, or a combination thereof. In general, functional fragments are at least 100 amino acids in length. In some embodiments of the present invention, functional fragments are defined as being at least 150 amino acids, at least 160 amino acids, at least 170 amino acids, at least 180 amino acids, and at least 190 amino acids in length.

In certain embodiments of the present invention, when a recombinant CP comprises a variant sequence, the variant sequence is at least about 70% identical to the parental (reference) sequence, for example, at least about 75% identical to the reference sequence. In some embodiments, the variant sequence is at least about 80%, at least about 85%, at least about 90%, at least about 95%, at least about 97% identical, at least about 98% identical to the reference sequence, or any amount therebetween. In one embodiment, the reference amino acid sequence is SEQ ID NO:1 (FIG. 1A).

In some embodiments of the invention, the PapMV CP used to prepare the recombinant PapMV CP is a genetically modified (i.e. variant) version of the PapMV CP. In some embodiments, the PapMV CP has been genetically modified to delete amino acids from the N- or C-terminus of the protein and/or to include one or more amino acid substitutions. In certain embodiments, the PapMV CP has been genetically modified to delete between about 1 and about 10 amino acids from the N- or C-terminus of the protein, for example between about 1 and about 5 amino acids.

In one embodiment, the PapMV CP has been genetically modified to remove one of the two methionine codons that occur proximal to the N-terminus of the wild-type protein and can initiate translation (i.e. at positions 1 and 6 of SEQ ID NO:1). Removal of one of the translation initiation codons allows a homogeneous population of proteins to be produced. The selected methionine codon can be removed, for example, by substituting one or more of the nucleotides that make up the codon such that the codon codes for an amino acid other than methionine, or becomes a nonsense codon. Alternatively all or part of the codon, or the 5′ region of the nucleic acid encoding the protein that includes the selected codon, can be deleted. In some embodiments, the PapMV CP has been genetically modified to delete the methionine at position 1, for example, by deleting between 1 and 5 amino acids from the N-terminus of the protein. In some embodiments, the genetically modified PapMV CP has an amino acid sequence substantially identical to SEQ ID NO:4 (FIG. 1C) and may optionally comprise a histidine tag of up to 6 histidine residues. In some embodiments, the PapMV CP has been genetically modified to include additional amino acids (for example between about 1 and about 8 amino acids) at the C-terminus. Introduction of such amino acids may, for example, result in the creation of one or more specific restriction enzyme sites in the encoding nucleotide sequence. In certain embodiments, the PapMV CP has an amino acid sequence substantially identical to SEQ ID NO:5 (FIG. 1D) with or without the histidine tag.

When the recombinant PapMV CP is prepared using a variant PapMV CP sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

In certain embodiments, the recombinant CP comprises a variant sequence having one or more non-conservative substitutions. Replacement of one amino acid with another having different properties may improve the properties of the CP. For example, as previously described, mutation of residue 128 of the CP improves assembly of the protein into VLPs (Tremblay et al., 2006, FEBS, 273:14-25). In some embodiments, therefore, the CP comprises a mutation at residue 128 in which the glutamic acid residue at this position is substituted with a neutral residue. In one embodiment, the glutamic acid residue at position 128 is substituted with an alanine residue.

Substitution of the phenylalanine residue at position F13 of the wild-type PapMV CP with another hydrophobic residue has been shown to result in a higher proportion of VLPs being formed when the recombinant protein is expressed than when the wild-type protein sequence is used. In the context of the present invention, the following amino acid residues are considered to be hydrophobic residues suitable for substitution at the F13 position: Ile, Trp, Leu, Val, Met and Tyr. In certain embodiments, the recombinant CP comprises a substitution of Phe at position 13 with Ile, Trp, Leu, Val, Met or Tyr. In one embodiment, the recombinant CP comprises a substitution of Phe at position 13 with Leu or Tyr.

In certain embodiments, mutation at position F13 of the CP may be combined with a mutation at position E128, a deletion at the N-terminus, a deletion at the C-terminus, or a combination thereof.

Likewise, the nucleic acid sequence encoding the PapMV CP used to prepare the recombinant PapMV CP need not correspond precisely to the parental reference sequence but may vary by virtue of the degeneracy of the genetic code and/or such that it encodes a variant amino acid sequence as described above. In certain embodiments of the present invention, therefore, the nucleic acid sequence encoding the variant CP is at least about 70% identical to the reference sequence. In some embodiments, the nucleic acid sequence encoding the recombinant CP is at least about 75% identical to a parental (reference) sequence, for example, at least about 80%, at least about 85%, at least about 90% identical to the reference sequence, or any amount therebetween. In one embodiment, the reference nucleic acid sequence is SEQ ID NO:2 (FIG. 1B).

### Antigenic Peptides

The recombinant PapMV CPs according to the present invention comprise one or more antigenic peptides fused to the CP within a predicted CP N-terminal surface-coil. Preferably, the antigenic peptides are derived from an influenza antigen. The antigenic peptides are selected such that they do not interfere with the ability of the recombinant CP to be expressed, or to self-assemble into VLPs, both of which can be tested by standard techniques, such as those described herein.

The antigenic peptides for fusion with the CP can vary in size, but in general are between about 3 amino acids and about 50 amino acids in length, for example between about 3 and about 40 amino acids, between about 3 and about 30 amino acids, between about 3 and about 25 amino acids, between about 3 and about 20 amino acids, between about 3 and about 15 amino acids, between about 3 and about 12 amino acids in length, or any amount therebetween. In some embodiments, the antigenic peptide is at least 5 amino acids in length, for example at least 6 or at least 7 amino acids in length and up to about 10, 11, 12, 15 or 20 amino acids in length, or any amount therebetween. In certain embodiments of the invention, the antigenic peptide is 25 amino acids or less in length, for example, 20 amino acids or less, 15 amino acids or less, 14 amino acids or less, 13 amino acids or less, 12 amino acids or less, with the lower end of the range being, for example, 3, 4, 5, 6, 7, 8, 9 or 10 amino acids. In certain embodiments, the antigenic peptide is about 5, 6, 7, 8, 9, 10, 11, 12 or 13 amino acids in length.

The antigens from which the antigenic peptides are derived may comprise epitopes recognised by surface structures on T cells, B cells, NK cells, macrophages, Class I or Class II APC (antigen presenting cell) associated cell surface structures, or a combination thereof. In certain embodiments, the antigenic peptide comprises a T-cell or CTL epitope. As is known in the art, T-cell epitopes and CTL epitopes are recognized and bound by T-cell receptors, and may be located in the inner, unexposed portion of the antigen, and become accessible to the T-cell receptors after proteolytic processing of the antigen. CTL epitopes may also be found on the surface of an antigen. Various T-cell epitopes and CTL epitopes associated with the influenza virus are known in the art. In some embodiments, the antigenic peptides selected for fusion with the PapMV CP comprise B-cell epitopes. As is known in the art, B-cell epitopes are recognized and bound by the B-cell receptor. Such epitopes are typically located on the surface of the antigen. Various B-cell epitopes associated with the influenza virus are known in the art.

In certain embodiments, the antigenic peptides can comprise a combination of T-cell epitopes or CTL epitopes and B-cell epitopes, for example, when the recombinant CPs comprise more than one antigenic peptide.

Known influenza virus antigens include, for example, those derived from the haemagglutinin (HA), neuramidase (NA), nucleoprotein (NP), M1 and M2 proteins. The sequences of these proteins are known in the art and are readily accessible from GenBank database maintained by the National Center for Biotechnology Information (NCBI). Suitable antigenic peptides of HA, NP and the matrix proteins include, but are not limited to, fragments comprising one or more of the haemagglutinin epitopes: HA 91-108, HA 307-319 and HA 306-324 (Rothbard, Cell, 1988, 52:515-523), HA 458-467 (J. Immunol. 1997, 159(10): 4753-61), HA 213-227, HA 241-255, HA 529-543 and HA 533-547 (Gao, al., J. Virol., 2006, 80:1959-1964); the nucleoprotein epitopes: NP 206-229 (Brett, 1991, J. Immunol. 147:984-991), NP335-350 and NP380-393 (Dyer and Middleton, 1993, In: Histocompatibility testing, a practical approach (Ed.: Rickwood, D. and Hames, B. D.) IRL Press, Oxford, p. 292; Gulukota and DeLisi, 1996, Genetic Analysis: Biomolecular Engineering, 13:81), NP 305-313 (DiBrino, 1993, PNAS 90:1508-12); NP 384-394 (Kvist, 1991, Nature 348:446-448); NP 89-101 (Cerundolo, 1991, Proc. R. Soc. Lon. 244:169-7); NP 91-99 (Silver et al, 1993, Nature 360: 367-369); NP 380-388 (Suhrbier, 1993, J. Immunology 79:171-173); NP 44-52 and NP 265-273 (DiBrino, 1993, ibid); and NP 365-380 (Townsend, 1986, Cell 44:959-968); the matrix protein (M1) epitopes: M1 2-22, M1 2-12, M1 3-11, M1 3-12, M1 41-51, M1 50-59, M1 51-59, M1 134-142, M1 145-155, M1 164-172, M1 164-173 (all described by Nijman, 1993, Eur. J. Immunol. 23:1215-1219); M1 17-31, M1 55-73, M1 57-68 (Carreno, 1992, Mol Immunol 29:1131-1140); M1 27-35, M1 232-240 (DiBrino, 1993, ibid.), M1 59-68 and M1 60-68 (Eur. J. Immunol. 1994, 24(3): 777-80); and M1 128-135 (Eur. J. Immunol. 1996, 26(2): 335-39).

Other antigenic regions and epitopes of the influenza virus proteins are known, for example, fragments of the influenza ion channel protein (M2), including the M2e peptide (the extracellular domain of M2). The sequence of this peptide is highly conserved across different strains of influenza. In certain embodiments of the invention, the antigenic peptide is derived from the M2e peptide. An example of a M2e peptide sequence is shown in Table 1 as SEQ ID NO:8. Variants of this sequence have been identified and some examples of such variants are also shown in Table 1.

TABLE 1
M2e Peptide and Variations Thereof

| Region | | SEQ ID |
|---|---|---|
| of M2 | Sequence | NO |
| 2-24 | SLLTEVETPIRNEWGCRCNDSSD | 8 |
| 2-24 | SLLTEVETPIRNEWGCRCNGSSD^{*} | 9 |
| 2-24 | SLLTEVETPTKNEWDCRCNDSSD^{*} | 10 |
| 2-24 | SLLTEVETPTRNGWECKCSDSSD^{‡} | 11 |
| 2-24 | SLLTEVETPTRNEWECRCSDSSD^{#} | 12 |

^{*}see U.S. Patent Application No. 2006/0246092
^{‡}A/equine/Massachussetts/213/2003 (strain H3N8)
^{#}A/Vietnam/1196/04 (strain H5N1)

In certain embodiments, the entire M2e sequence may be used. In some embodiments, preferably a partial M2e sequence is used, for example, a partial sequence that is conserved across M2e variants, such as fragments comprising the region defined by amino acids 2 to 10, or fragments comprising the region defined by amino acids 6 to 13.

In certain embodiments, the antigenic peptide comprises a peptide derived from M2e that includes the region defined by amino acids 6 to 13, or a fragment thereof. The sequence of the region of M2e defined by amino acids 6 to 13 can be defined as:

[SEQ ID NO: 95]
E-V-X1-T-X2-X3-X4-X5,

where

X1 is E or D;

X2 is P or L;

X3 is T or I;

X4 is R or K, and

X5 is N, S or K.

For example, the epitope EVETPIRN [SEQ ID NO: 13] is found in 84% of human influenza A strains available in GenBank. Variants of this sequence that have also been identified include EVETLTRN [SEQ ID NO:14] (9.6%), EVETPIRS [SEQ ID NO:15] (2.3%), EVETPTRN [SEQ ID NO:16] (1.1%), EVETPTKN [SEQ ID NO:17] (1.1%) and EVDTLTRN [SEQ ID NO:18], EVETPIRK [SEQ ID NO:19] and EVETLTKN [SEQ ID NO:20] (0.6% each) (see Zou, et al., 2005, Int Immunopharmacology, 5:631-635; Liu et al. 2005, Microbes and Infection, 7:171-177).

In certain embodiments, therefore, the antigenic peptide is an M2e-derived peptide comprising the general sequence E-V-X1-T-X2-X3-X4-X5 [SEQ ID NO:95], such as those exemplified above, or a fragment thereof. Exemplary fragments include those having the sequence: V-X1-T-X2-X3-X4-X5 [SEQ ID NO:96], for example, VETPIRN [SEQ ID NO:97], VETLTRN [SEQ ID NO:98], VETPIRS [SEQ ID NO:99], VETPTRN [SEQ ID NO:100], VETPTKN [SEQ ID NO:101], VDTLTRN [SEQ ID NO:102], VETPIRK [SEQ ID NO:103] and VETLTKN [SEQ ID NO:104].

In certain embodiments, the antigenic peptide selected for fusion with the PapMV CP comprises a portion of the M2e peptide between about 5 and about 12 amino acids in length, for example, between about 5 and about 10 amino acids in length. Suitable portions of the M2e peptide include those described above. In some embodiments, the antigenic peptide comprises a portion of the M2e peptide between about 5 and about 12 amino acids in length, for example, between about 5 and about 10 amino acids in length. In some embodiments, the antigenic peptide is less than 10 amino acids in length and comprises a peptide of general sequence SEQ ID NO:95 or 96, for example, the sequence EVETPIRNE [SEQ ID NO: 21] or VETPIRN [SEQ ID NO:22]. In certain embodiments, the antigenic peptide may consist essentially of the sequence EVETPIRNE [SEQ ID NO: 21] or VETPIRN [SEQ ID NO:22].

Exemplary, non-limiting examples of recombinant PapMV CPs comprising an M2e peptide include PapMV CP fusions comprising an amino acid sequence as set forth in SEQ ID NO:23 from amino acid 1-224; in SEQ ID NO:24 from amino acid 1-222; in SEQ ID NO:25 from amino acid 1-221; in SEQ ID NO:26 from amino acid 1-219; in SEQ ID NO:27 from amino acid 1-224; and in SEQ ID NO:28 from amino acid 1-222, as well as those comprising the amino acid sequence as set forth in any one of SEQ ID NOs:23-28.

### Fusion of Antigenic Peptides Within PapMV CP Surface-Coil Region

In accordance with certain embodiments of the present invention, the recombinant PapMV CPs comprise one or more antigenic peptides fused within the predicted random coil within 13 amino acids of the N-terminus of the CP (see FIG. 16 in which the random coil regions at the N- and C-termini of the CP are marked in bold).

Accordingly, some embodiments of the invention provide for recombinant PapMV CPs in which one or more antigenic peptides are fused after a position corresponding to amino acid 1, 2, 3, 4, 5, 6, 7, 8, 9, 10, 11 or 12 of the PapMV CP sequence shown in SEQ ID NO:1. In some embodiments, the PapMV CPs used for the preparation of the fusion proteins are variants in which the methionine at position 1 of SEQ ID NO:1 has been deleted or substituted such that the first residue of the expressed CP is the methionine at position 5 of SEQ ID NO:1. In such embodiments, fusion of the antigenic peptides after a position corresponding to amino acid 6, 7, 8, 9, 10, 11 or 12 of the PapMV CP sequence shown in SEQ ID NO:1 are contemplated. In certain embodiments, the antigenic peptide(s) may be fused after a position corresponding to amino acid 6, 7, 8, 9 or 10 of the PapMV CP sequence shown in SEQ ID NO:1. In certain embodiments, the antigenic peptide(s) may be fused after a position corresponding to amino acid 6, 7 or 10 of the PapMV CP sequence shown in SEQ ID NO:1.

In certain embodiments, the PapMV CP used for the preparation of the fusion proteins has a sequence as set forth in SEQ ID NO:4 (FIG. 1C), and the one or more antigenic peptides are fused after amino acid 1, 2, 3, 4, 5, 6, 7 or 8 of SEQ ID NO:4. In some embodiments, the fusion protein may comprise one or more antigenic peptides fused after amino acid 2, 3, 4, 5 or 6 of the PapMV CP sequence shown in SEQ ID NO:4. In some embodiments, the fusion protein may comprise one or more antigenic peptides fused after amino acid 2, 3, 4, 5 or 6 of the PapMV CP sequence shown in SEQ ID NO:4. In some embodiments, the fusion protein may comprise one or more antigenic peptides fused after amino acid 2, 3 or 6 of the PapMV CP sequence shown in SEQ ID NO:4.

Certain embodiments relate to fusion of an M2e-derived peptide after a position corresponding to amino acid 6, 7 or 10 of the PapMV CP sequence shown in SEQ ID NO:1, for example, after amino acid 2, 3 or 6 of the PapMV CP sequence shown in SEQ ID NO:4. The M2e-derived peptide may be, for example, between about 5 and about 10 amino acids in length and comprise a sequence as outlined above.

Some embodiments relate to recombinant CPs which further comprise an antigenic peptide fused after amino acid 185, 186, 187, 188, 189, 190, 191 or 192 of the PapMV CP sequence shown in SEQ ID NO:1, and/or an antigenic peptide fused within one of the other predicted random coils located within the 30 C-terminal amino acids of the CP and/or one or more antigenic peptides fused at the C-terminus of the CP.

Some embodiments relate to recombinant CPs which further comprise an antigenic peptide fused after amino acid 197, 198, 199, 200, 201, 202, 203, 204, 205, 206, 207, 208, 209, 210, 211, 212, 213 or 214 of the PapMV CP sequence shown in SEQ ID NO:1, and/or an antigenic peptide fused after amino acid 185, 186, 187, 188, 189, 190, 191 or 192 of the PapMV CP sequence shown in SEQ ID NO:1 and/or one or more antigenic peptides fused at the C-terminus of the CP.

In certain embodiments, the recombinant CPs comprise one antigenic peptide fused to the CP within a single CP surface-coil region, or alternatively may comprise one antigenic peptide fused within each of one or more CP surface-coil regions, or they may comprise one antigenic peptide fused within each of two or more CP surface-coil regions. Optionally, the recombinant CPs may further comprise one or a plurality of antigenic peptides fused at the C-terminus of the CP.

In some embodiments, the recombinant PapMV CPs comprise more than one copy of the same antigenic peptide fused to the CP within one or more CP surface-coil sites, for example, more than one copy of the same antigenic peptide can be fused within a single CP surface-coil site or more than one copy of the same antigenic peptide can be fused within each of one or more CP surface-coil sites. Optionally, the recombinant CPs may further comprise one or a plurality of antigenic peptides fused at the C-terminus of the CP.

In those embodiments where more than one antigenic peptide is fused to the CP, the antigenic peptides may be the same or each antigenic peptide may be different.

In those embodiments in which multiple copies of an antigenic peptide are fused at one site in the CP, the overall length of the insertion is generally less than about 50 amino acids, for example, 40 amino acids or less, 35 amino acids or less, 30 amino acids or less, 25 amino acids or less, 20 amino acids or less, or 15 amino acids or less.

In certain embodiments, the selected antigenic epitopes are inserted into the PapMV CP together with one or more flanking sequences to assist with presentation of the antigenic peptide. Such flanking sequences may be present on one or both sides of the antigenic peptide. When the flanking sequences are on both sides, the amino acid sequences of these flanking sequences may be the same or they may be different. Flanking sequences, when used, are typically between about 1 and about 10 amino acids in length, for example, between about 2 and about 10 amino acids, between about 2 and about 9 amino acids, between about 2 and about 8 amino acids, between about 2 and about 7 amino acids, between about 2 and about 6 amino acids, between about 2 and about 5 amino acids, or between about 3 and about 5 amino acids. Flanking sequences can be particularly useful in conjunction with antigenic peptides comprising CTL epitopes. In general, in those embodiments which employ flanking sequences, the overall length of the inserted sequence is kept to less than about 50 amino acids, for example, 40 amino acids or less, 35 amino acids or less, 30 amino acids or less, 25 amino acids or less, 20 amino acids or less, or 15 amino acids or less.

### Preparation of the Recombinant PapMV Coat Proteins

The present invention provides recombinant PapMV CPs comprising one or more antigenic peptides. Methods of genetically fusing the antigenic peptides to the CP are known in the art and include those described below and in the Examples. Methods of chemically cross-linking antigenic peptides to proteins are also well known in the art and can be employed, where appropriate.

### Recombinant PapMV Coat Proteins

The recombinant PapMV CPs according to the invention can be readily prepared by standard genetic engineering techniques by the skilled worker provided with the sequence of the wild-type or parental protein. Methods of genetically engineering proteins are well known in the art (see, for example, Ausubel et al. (1994 & updates) Current Protocols in Molecular Biology, John Wiley & Sons, New York), as are the amino acid and nucleotide sequences of the wild-type PapMV CP (see SEQ ID NOs:1 and 2).

When necessary, isolation and cloning of the nucleic acid sequence encoding the wild-type protein can be achieved using standard techniques (see, for example, Ausubel et al., ibid.). For example, the nucleic acid sequence can be obtained directly from the PapMV by extracting RNA by standard techniques and then synthesizing cDNA from the RNA template (for example, by RT-PCR). PapMV can be purified from infected plant leaves that show mosaic symptoms by standard techniques.

Alternatively, the nucleic acid sequence encoding the recombinant CP may be prepared by known in vitro techniques (see, for example, Ausubel et al. ibid.).

The nucleic acid sequence encoding the CP is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses.

Alternatively, the nucleic acid sequence encoding the CP can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

The recombinant PapMV CPs are engineered to insert the one or more antigenic peptides at the desired site, to produce the recombinant CP fusion. Methods for making fusion proteins are well known to those skilled in the art. DNA sequences encoding a fusion protein can be inserted into a suitable expression vector as noted above.

One of ordinary skill in the art will appreciate that the DNA encoding the CP or fusion protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the coat or fusion protein. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a recombinant CP. One skilled in the art will appreciate that selection of suitable regulatory elements is dependent on the host cell chosen for expression of the genetically engineered CP and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed protein. such heterologous nucleic acid sequences can be located at the carboxyl terminus or the amino terminus of the CP. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed CP prior to use according to methods known in the art. Alternatively, the amino acids corresponding to expression of heterologous nucleic acid sequences can be retained on the CP if they do not interfere with its multimerization.

In some embodiments of the present invention, the CP is expressed as a histidine tagged protein. The histidine tag can be located at the carboxyl terminus or the amino terminus of the CP. In certain embodiments, the histidine tag is located at the carboxyl terminus of the CP.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the recombinant CP will be dependent upon the vector chosen. Examples of host cells include, but are not limited to, bacterial, yeast, insect, plant and mammalian cells. The precise host cell used is not critical to the invention. The recombinant CPs can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; or insect cells). In one embodiment, the recombinant CPs are expressed in prokaryotic cells.

If desired, the recombinant CPs can be purified from the host cells by standard techniques known in the art (see, for example, in Current Protocols in Protein Science, ed. Coligan, J. E., et al., Wiley & Sons, New York, N.Y.) and optionally may be sequenced by standard peptide sequencing techniques using either the intact protein or proteolytic fragments thereof to confirm the identity of the protein.

### Preparation of VLPs

Recombinant PapMV CPs useful in the context of the present invention are capable of assembly into VLPs. In certain embodiments of the invention, the recombinant CPs are allowed to assemble into VLPs within the host cell expressing the CP. The VLPs can be isolated from the host cells by standard techniques, such as those described in Denis et al. 2007, Virology, 363:59-68; Denis et al., 2008, Vaccine, 26; 3395-3403, and Tremblay et al., 2006, FEBS, 273:14-25. In general, the isolate obtained from the host cells contains a mixture of VLPs, discs, and less organised forms of the CP (for example, monomers and dimers).

In certain embodiments, PapMV VLPs may also be prepared isolating low molecular weight forms of the recombinant PapMV CP (primarily, but not exclusively, monomers) from the host cell and allowing the CP to assemble in vitro as described in International Patent Application No. PCT/CA2012/050279 (WO 2012/155262). In accordance with this method, recombinant CP and ssRNA are combined at a protein:RNA ratio of between about 1:1 and 50:1 by weight, at a pH between about 6.0 and about 9.0, and a temperature between about 2° C. and about 37° C., for a time sufficient to allow assembly of VLPs. The VLPs are subsequently treated with nuclease to remove any RNA protruding from the particles, and then optionally separated from other process components. This in vitro method can provide for up to about 80% of the recombinant CP being converted into VLPs.

The VLPs can be prepared from a plurality of recombinant CPs having identical amino acid sequences, such that the final VLPs comprise identical CP subunits, or the VLPs can be prepared from a plurality of recombinant CPs having different amino acid sequences, such that the final VLPs comprise variations in its CP subunits.

When required, the VLPs can be separated from the other CP components by, for example, ultracentrifugation or gel filtration chromatography (for example, using Superdex G-200) to provide a substantially pure VLP preparation. In this context, by “substantially pure” it is meant that the preparation contains 70% or greater of VLPs, for example, 75% or greater, 80% or greater, 85% or greater, or any amount therebetween. While it is contemplated that a mixture of the various forms of CP can be used in the final vaccine compositions, it is preferred that substantially pure VLP preparations are employed.

In certain embodiments, preparations of recombinant CPs that contain both VLPs and discs are employed. These may be prepared for example by utilizing the expressed recombinant CP, which comprises VLPs and discs, with or without dialysis and/or concentration steps.

The VLPs can be further purified by standard techniques, such as chromatography, to remove contaminating host cell proteins or other compounds, such as LPS. In one embodiment of the present invention, the VLPs are purified to remove LPS.

### Characteristics of Recombinant Coat Proteins

The recombinant CPs can be analyzed for their ability to self-assemble into a VLP by standard techniques, for example, by visualising the purified recombinant protein by electron microscopy (see, for example, the Examples provided herein). VLP formation may also be determined by ultracentrifugation, and circular dichroism (CD) spectrophotometry may be used to compare the secondary structure of the recombinant proteins with the WT virus if desired. The size of the VLPs can be assessed by dynamic light scattering (DLS).

Stability of the VLPs can be determined if desired by techniques known in the art, for example, by SDS-PAGE and proteinase K degradation analyses. Thermostability of the VLPs may be assessed, for example, by CD spectrophotometry and/or DLS (as described in the Examples).

In certain embodiments of the present invention, the recombinant PapMV VLPs are stable at elevated temperatures. In some embodiments, the recombinant PapMV VLPs are stable at elevated temperatures and can be stored easily at room temperature. In some embodiments, the recombinant PapMV VLPs are stable at temperatures of 25° C. or greater, for example 30° C. or greater, 35° C. or greater, or 37° C. or greater, as assessed by dynamic light scattering (DLS), for example.

The PapMV VLPs formed from recombinant PapMV CPs comprise a long helical array of CP subunits. The wild-type virus comprises over 1200 CP subunits and is about 500 nm in length. PapMV VLPs that are either shorter or longer than the wild-type virus can still, however, be effective. In one embodiment of the present invention, VLPs formed from recombinant PapMV CPs comprise at least 40 CP subunits. In another embodiment, VLPs formed from recombinant PapMV CPs comprise between about 40 and about 1600 CP subunits. In an alternative embodiment, VLPs formed from recombinant PapMV CPs are at least 40 nm in length. In another embodiment, the VLP is between about 40 nm and about 600 nm in length.

### Evaluation of Efficacy

The efficacy of the VLPs comprising the recombinant PapMV CPs in inducing an immune response to the antigenic peptide comprised by the recombinant CP can be assessed by various standard in vitro and in vivo techniques known in the art.

For example, for in vivo testing, groups of test animals (such as mice) can be inoculated with the VLPs by standard techniques. Control groups comprising non-inoculated animals and/or animals inoculated with the antigenic peptide, a commercially available vaccine, or other positive control, are set up in parallel. Blood samples collected from the animals pre- and post-inoculation are then analyzed for an antibody response to the antigen. Suitable tests for the antibody response include, but are not limited to, Western blot analysis and Enzyme-Linked Immunosorbent Assay (ELISA).

In order to further evaluate the efficacy of the VLPs comprising the recombinant PapMV CPs as vaccines, challenge studies can be conducted. Animals are inoculated as described above and after an appropriate period of time post-vaccination, the animals are challenged with the disease causing agent of interest, for example an influenza virus. Blood samples can be collected and analyzed. The animals can also be monitored for development of other conditions associated with infection including, for example, body temperature, weight, and the like. In certain cases, such as, for example when certain strains of influenza virus are used, survival is also a suitable marker. The extent of infection may also be assessed by measurement of lung viral titer using standard techniques after sacrifice of the animal.

Cellular immune responses can also be assessed if desired by techniques known in the art. For example, through processing and cross-presentation of an epitope expressed on a PapMV VLP to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines.

### Pharmaceutical Compositions and Vaccine Formulations

Certain embodiments of the present invention relate to pharmaceutical compositions comprising the VLPs comprising recombinant PapMV CPs, together with one or more pharmaceutically acceptable carriers, diluents and/or excipients. If desired, other active ingredients, adjuvants and/or immunopotentiators may be included in the compositions. In certain embodiments, the pharmaceutical compositions may be included in, or formulated as, vaccines.

The pharmaceutical compositions and/or vaccines can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the pharmaceutical composition to the mucous membranes of the nasal passage or nasal cavity of the subject. In certain embodiments, the compositions are formulated for parenteral administration or for administration by inhalation or spray, for example by an intranasal route. In some embodiments, the compositions are formulated for parenteral administration.

The compositions preferably comprise an effective amount of the VLPs comprising the recombinant PapMV CPs. The term “effective amount” as used herein refers to an amount of the VLPs required to induce a detectable immune response. The effective amount of the VLPs for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the present invention, the unit dose comprises between about 10 μg to about 10 mg of protein. In another embodiment, the unit dose comprises between about 10 μg to about 5 mg of protein. In a further embodiment, the unit dose comprises between about 40 μg to about 2 mg of protein. One or more doses may be used to immunise the animal, and these may be administered on the same day or over the course of several days or weeks. In certain embodiments, a single dose of the vaccine composition is sufficient to provide a protective effect. In some embodiments, one or more additional booster shots at appropriate interval(s) are also contemplated.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavoring agents, coloring agents and preserving agents in order to provide pharmaceutically elegant and palatable preparations. Tablets contain the VLPs in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the VLPs in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the VLPs in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the VLPs in admixture with one or more dispersing or wetting agents, suspending agents and/or preservatives. Suitable dispersing or wetting agents and suspending agents are exemplified by those already mentioned above.

Compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the compositions of the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Further, one or more compounds having adjuvant activity may be optionally added to the composition. Suitable adjuvants include, for example, alum adjuvants (such as aluminium hydroxide, phosphate or oxide); oil-emulsions (e.g. of Bayol F® or Marcol52®); saponins, or vitamin-E solubilisate. Virosomes are also known to have adjuvant properties (Adjuvant and Antigen Delivery Properties of Virosomes, Glück, R., et al., 2005, Current Drug Delivery, 2:395-400) and can be used in conjunction with the multimers according to the invention.

As previously demonstrated, PapMV and PapMV VLPs have adjuvant properties. Accordingly, in one embodiment of the invention, the compositions may comprise additional PapMV or PapMV VLPs as an adjuvant. In some embodiments, use of PapMV or PapMV VLPs may provide advantages over commercially available adjuvants in that it has been observed that PapMV or PapMV VLPs do not cause obvious local toxicity when administered by injection (see, for example, International Patent Publication No. WO2008/058396).

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Applications & Uses

A number of applications and uses of the VLPs comprising recombinant PapMV CPs are contemplated by the present invention. Certain embodiments of the invention relate to the use of the VLPs to induce a protective immune response against an influenza virus. Methods of immunizing a subject against influenza infection using the VLPs are also provided in certain embodiments. Some embodiments of the invention relate to vaccines comprising the VLPs for prophylactic administration to a subject to reduce the risk of contracting influenza.

Some embodiments of the invention thus relate to the use of the VLPs for the preparation of medicaments, including vaccines, and/or pharmaceutical compositions.

Certain embodiments of the present invention relate to the use of the VLPs comprising the recombinant CP for eliciting a humoral immune response against an influenza virus in a subject, for example, in some embodiments, the recombinant CPs comprise antigenic peptides that include a B-cell epitope and are suitable for use to elicit a humoral immune response in a subject.

In some embodiments, the recombinant CPs comprise antigenic peptides that include a T-cell epitope or a CTL epitope and are suitable for use as vaccines for eliciting a cellular immune response in a subject.

Certain embodiments of the invention relate to vaccines comprising the VLPs to provide protection against more than one strain of influenza virus.

Certain embodiments of the invention relate to the use of the VLPs to induce a protective immune response in humans. Some embodiments of the invention relate to the use of the VLPs to induce a a protective immune response in non-human animals, including domestic and farm animals. The administration regime for the VLPs need not differ from any other generally accepted vaccination programs. A single administration of the VLPs in an amount sufficient to elicit an effective immune response may be used or, alternatively, other regimes of initial administration of the recombinant VLPs followed by boosting, once or more than once, with the appropriate antigen alone or with the VLPs may be used. Similarly, boosting with either the appropriate antigen alone or with the VLPs may occur at times that take place well after the initial administration if antibody titers fall below acceptable levels. Appropriate dosing regimens can be readily determined by the skilled practitioner.

### Pharmaceutical Packs & Kits

Some embodiments of the present invention relate to pharmaceutical packs or kits comprising VLPs comprising recombinant CP. Kits comprising nucleic acids encoding one or more recombinant CPs are also provided. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the VLPs, or for the preparation of VLPs from the nucleic acids encoding the recombinant CP.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, nasal spray device, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

In the Examples section, fusions of antigenic peptides to various positions in the PapMV CP are described. These positions are referred to in the Examples based on the position within the N-terminal deletion sequence shown in FIG. 1C (SEQ ID NO:4). Positions referred to as positions 8, 29, 80, 118, 130, 158 and 183 (with reference to SEQ ID NO:4) are analogous to positions 12, 33, 84, 122, 134, 162 and 187, respectively, of the wild-type sequence [SEQ ID NO:1; FIG. 1A].

### Example 1

### Preparation and Evaluation of Recombinant PapMV Coat Proteins Fused to Influenza M2e Peptide

The ability of the PapMV coat protein (CP) to be expressed when fused with the HA11 epitope at different regions of the CP was investigated and the results reported in Rioux et al. (2012, PLoS ONE, 7(2):e31925). It was found that fusion of the HA11 peptide after amino acid 80 or after amino acid 130 of the PapMV CP [SEQ ID NO:4] led to an unstable protein and while fusion of the HA11 peptide after amino acids 29, 118 or 158 led to production of recombinant fusion proteins, this was at low yield. Fusion of the HA11 peptide after amino acids 8, 183, or at the C-terminus of the PapMV CP, however, resulted in the production of recombinant CPs with high yield. These recombinant proteins were also able to self-assemble into VLPs and the VLPs comprising the HA11 fusion after amino acid 8 triggered an immune response to the HA peptide in mice.

VLPs harbouring a fusion of the M2e peptide (28 a.a.) to the C-terminus of the PapMV CP have been previously described (Denis et al., 2008, Vaccine 26:3395-3403) and shown to trigger an immune response to the M2e peptide and a level of protection to influenza challenge in mice, which was further improved by addition of PapMV VLPs (without the fused peptide). Analysis of the PapMV-M2e-C VLPs by dynamic light scattering (DLS) showed that these VLPs are unstable at temperatures exceeding 30° C. suggesting that the of fusion at the C-terminus for this peptide is not optimal (see Rioux et al., ibid.).

In an attempt to increase the stability of PapMV VLPs harbouring the M2e peptide, Rioux et al. (ibid.) also prepared a construct containing the M2e peptide (SLLTEVETPIRNEWGCRCNDSS; SEQ ID NO:7) fused after position 8 of the CP [SEQ ID NO:4]. While the recombinant CP appeared stable, it failed to assemble into VLPs.

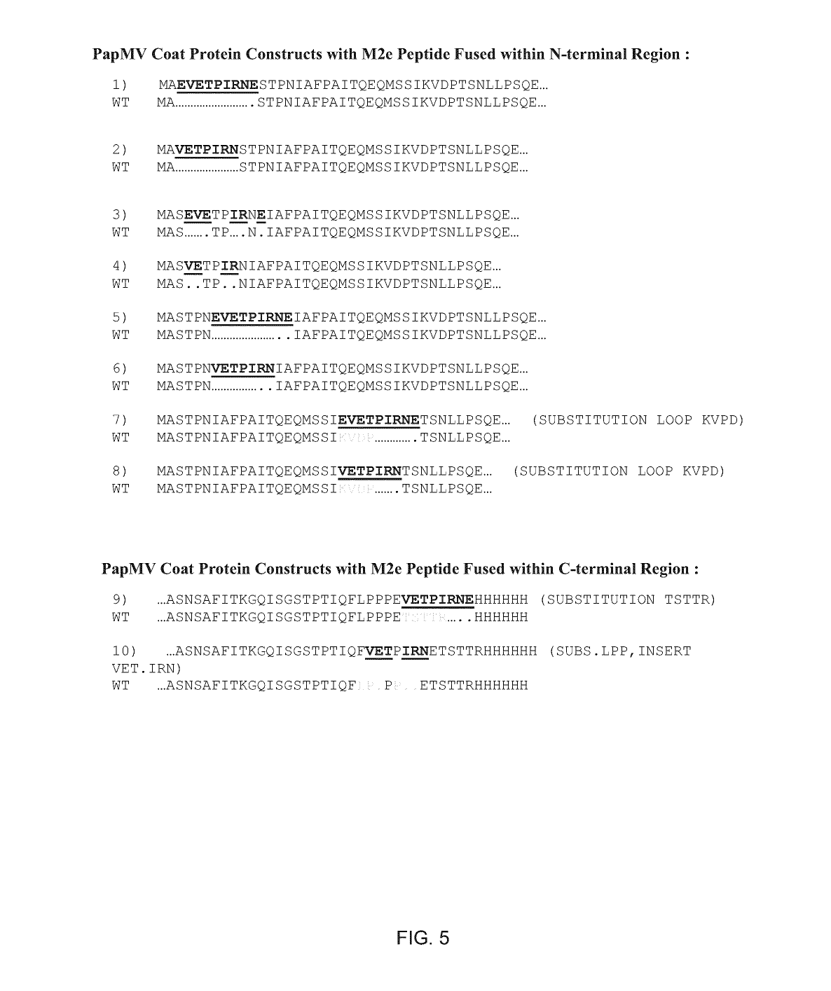

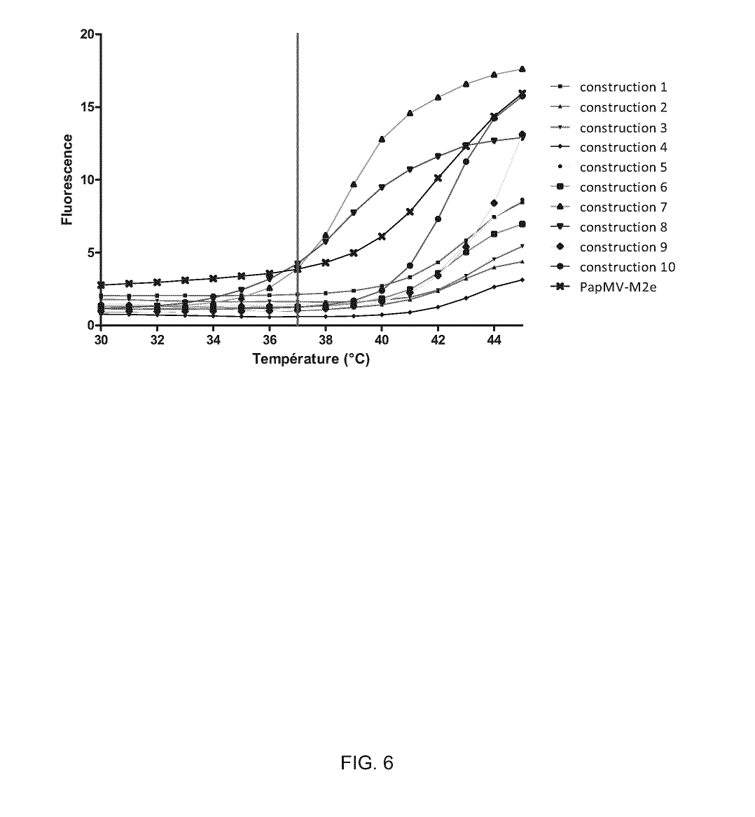

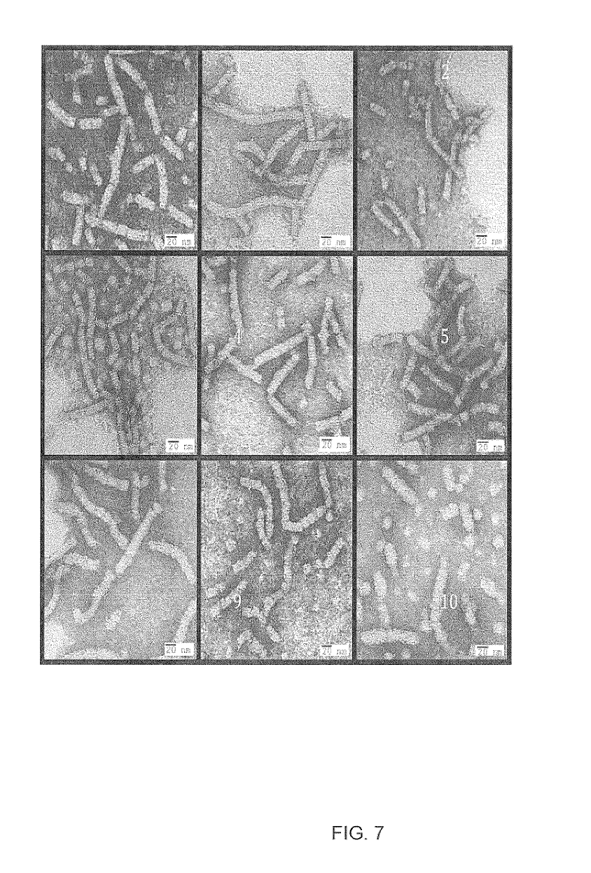

It is predicted that fusion of a shorter M2e peptide will have a lesser impact on the self-assembly of the CP and allow for production of stable and immunogenic VLPs. In order to investigate this prediction, fusion of a central portion of the M2e peptide was undertaken, specifically using the sequences EVETPIRNE [SEQ ID NO: 21] and VETPIRN [SEQ ID NO:22]. Ten constructs comprising PapMV coat protein fused to the M2e derived peptides (EVETPIRNE [SEQ ID NO:21] or VETPIRN [SEQ ID NO:22]) were prepared. Eight of the constructs (constructs #1-8) comprised a fusion in the N-terminal region of the coat protein and two (constructs #9 and 10) comprised a fusion in the C-terminal region (see Table 2 and FIG. 5). Eight of the constructs (constructs #1-6, 9 and 10) showed suitable size, thermostability and ability to form VLPs (see FIGS. 6 and 7). These eight constructs were injected into mice to evaluate their ability to raise an immune response. With the exception of constructs #9 and 10, the constructs were surprisingly able to produce a strong humoral response after only one immunization. In contrast, an increase in the level of antibodies after the second immunization was observed only for construct #1. From these results it appears that, for the M2e peptide, the placement of the fusion and the length of the peptide may cause structural changes in the coat protein that affect the stimulation of the immune system by the constructs.

The results showed that construct #1 resulted in the best humoral response and would be a suitable candidate for use in a Universal Influenza A vaccine.

TABLE 2
Size of VLPs assessed by dynamic light scattering (DLS)

| | M2e Peptide Sequence | Position of M2e | Size |
|---|---|---|---|
| Construct | [SEQ ID NO] | Peptide Insertion* | (nm) |
| Construct # 1 | EVETPIRNE [21] | After amino acid 2 | 103.80 |
| Construct # 2 | VETPIRN [22] | After amino acid 2 | 88.81 |
| Construct # 3 | EVETPIRNE [21] | After amino acid 3 | 64.49 |
| Construct # 4 | VETPIRN [22] | After amino acid 3 | 65.64 |
| Construct # 5 | EVETPIRNE [21] | After amino acid 6 | 56.43 |
| Construct # 6 | VETPIRN [22] | After amino acid 6 | 79.86 |
| Construct # 7 | EVETPIRNE [21] | After amino acid 20 | 24.25 |
| Construct # 8 | VETPIRN [22] | After amino acid 20 | 105.80 |
| Construct # 9 | EVETPIRNE [21] | C-terminus | 78.93 |
| Construct # 10 | VETPIRN [22] | C-terminus | 72.00 |
| PapMV-M2e^{†} | SLLTEVETPIRNEWGC | C-terminus | 53.13 |
| | RCNDSSD [8] | | |

*With reference to SEQ ID NO: 4.
^{†}see Denis et al. 2008, Vaccine, 26: 3395-3403.

### Methods:

The recombinant proteins were expressed in E. coli BL219DE3 using the pET3D vector inducible with 1 mM IPTG. The recombinant protein was purified as previously described in Denis et al., 2008, Vaccine, 26; 3395-3403. Protein expression was conducted for 16 hours at 25 degrees Celsius. The bacterial pellet was lysed using a French press, clarified by centrifugation (10000 g for 20 min) and loaded on a Ni²⁺column (IMAC). The bound recombinant protein was eluted from the IMAC column with 500 mM to 1M imidazole. Detergents (TX-100 1 to 2%+Zwittergent 1%) were used to remove the LPS. After elution, imidazole was removed by dialysis in 10 mM Tris HCl pH8. The PapMV VLPs were recovered after dialysis by ultracentrifugation for 90 min at 100 000 g. The pellet containing the VLPs was resuspended in 10 mM Tris HCl pH8.0 at 1 mg/mL.

To confirm that the proteins had correctly formed VLPs, the partial dematuration of the protein was measured by binding of a dye (Sypro Orange) to hydrophobic residues that become exposed when the temperature reaches the start of the denaturing point for the protein. For inclusion in further analysis, the VLPs needed to be stable at 37 degrees Celsius. Several construct were found to be stable and included in further evaluations (FIG. 6). Observation by electron microscopy confirmed the rod shape structure of the different constructs (FIG. 7).

The immune response towards the M2e peptide was evaluated in vivo by intramuscular immunization of mice with the VLPs with either one or two immunizations. The humoral response (total IgG and IgG2a) was monitored at day 14 (after one immunization) and at day 28 (after 2 immunizations).

### Results:

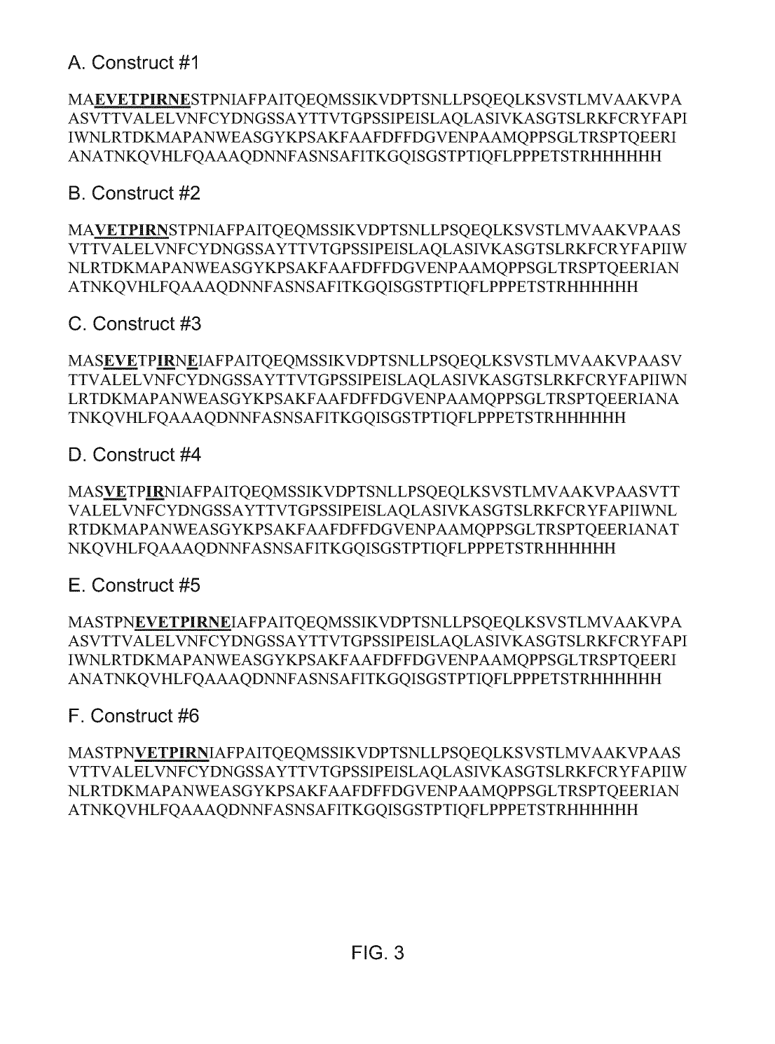

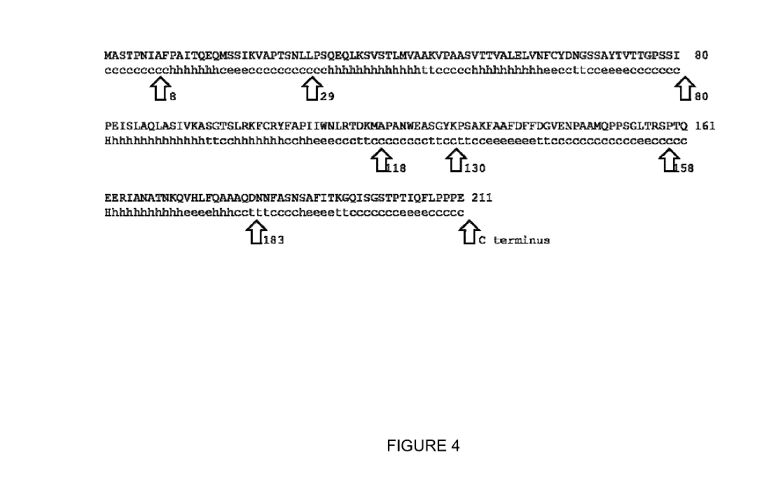

The results of the evaluation of the constructs are shown in Table 7 and FIGS. 6-8. The sequences for constructs #1-6 are provided in FIG. 3 [SEQ ID NOs: 23-28].

FIG. 6 shows that constructs #1, 2, 3, 4, 5 and 9 are thermally stable up to 40° C.; constructs #6 and 10 are thermally stable up to 38° C., and PapMV-M2e is thermally stable up to 37° C. Constructs #7 and 8 become unstable at 32° C. to 34° C. respectively.

FIG. 7 shows that all the thermostable constructs (#1, 2, 3, 4, 5, 6, 9 and 10) form VLPs and are similar in size and shape.

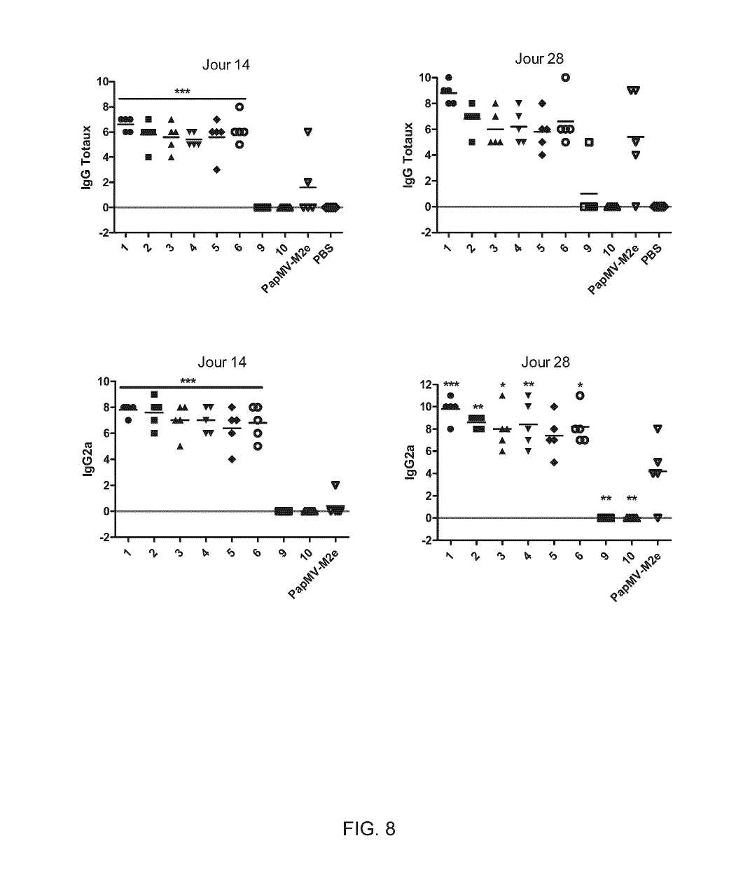

FIG. 8 shows that, with the exception of constructs #9 and 10, all the thermostable constructs were able to produce a strong humoral response after the first immunization. The level of IgG antibody increased following the second immunization only for construct #1 (A). An increase in the level of antibody subtype IgG2a following the second immunization was observed for the majority of the groups (B).

### Discussion:

All constructs formed VLPs with the exception of construct #7, which formed disks. This is likely due to the position of the M2e peptide insertion, which may have changed the structure of the coat protein preventing its ability to multimerize and form VLPs.

For the induction of an immune response against the fusion peptide, injected proteins must be heat-stable at the internal temperature of the animal (37° C.—represented by the red bar in FIG. 6). The reaction with Sypro Orange allows the point at which the protein denatures to be identified by measuring the increase in fluorescence. Only two constructs, #7 and 8, denatured before reaching the target temperature.

To confirm the results obtained by DLS shown in Table 2 and to visualize the morphology of the particles, transmission electron microscopy images were obtained. Rods were observed for all thermostable constructs (#1, 2, 3, 4, 5, 6, 9 and 10) (FIG. 7). Rod-shaped VLPs are desirable as they are more immunogenic.

To compare the immunogenicity of each of the fusions, ELISA were performed against the M2e peptide using sera collected 14 days after each of the immunizations. Contrary to the expected results, the level of IgG antibody did not increase following the second immunization (FIG. 8A), except for construct #1. An increase in the level of antibody subtype IgG2a following the second immunization was observed for the majority of the groups (FIG. 8B).

To conclude, constructs #1 to 6, 9 and 10 met all structural selection criteria, but only constructs #1 to 6 were able to produce an M2e epitope-specific immune response. Construct #1 produced a better humoral response against the M2e peptide suggesting that fusion of the M2e peptide after position 2 of SEQ ID NO:4 caused less structural changes in the coat protein. The results also suggested that the longest epitope, EVETPIRNE [SEQ ID NO:21] as used in construct #1, generates more avid antibodies to the native M2e antigen.

### Example 2

### Biochemical and Biophysical Characterization of Recombinant PapMV CP Fused at Different Positions to a Salmonella Porin Antigenic Peptide

Recombinant CP fusion proteins were prepared using a B-cell epitope from Salmonella typhi: the loop 6 peptide derived from the OmpC porin (GTSNGSNPSTSYGFAN [SEQ ID NO:29]). The loop 6 epitope is derived from the OmpC porin, a membrane bound protein of S. typhi (the agent of typhoid fever) that is exposed on the surface of the bacterium and has been shown to be involved in protective mechanisms elicited by immunization with porins (Paniagua-Solís et al., 1996, FEMS Microbiol Lett., 14:31-6). These regions are only present in S. typhi porins, therefore, no cross-reactivity with porins from other gram-negative bacteria has been found.

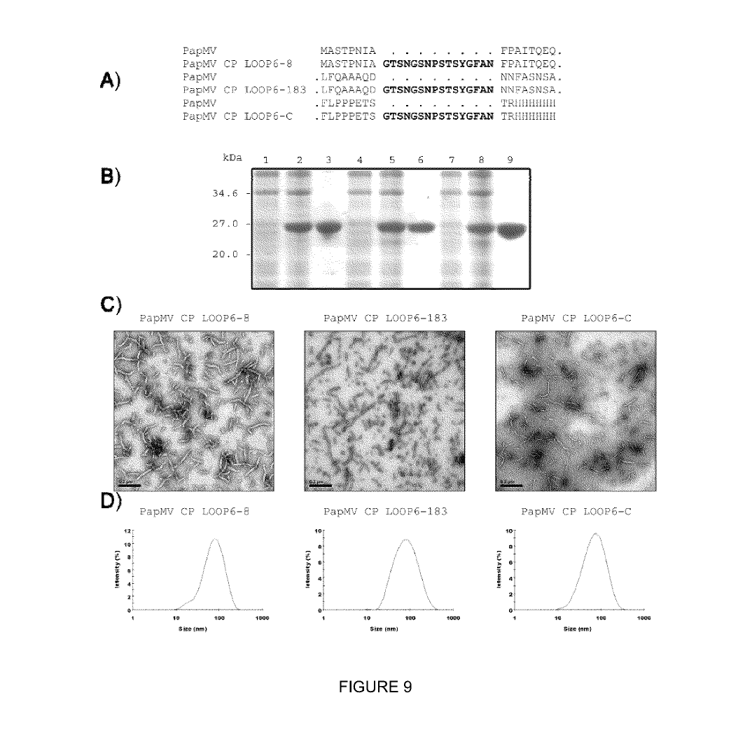

Proteins harboring a fusion of the loop 6 peptide at position 8, position 183, or at the C-terminus of PapMV CP were produced (see FIG. 9A). The respective fusions were named PapMV CP Loop6-8, PapMV CP Loop6-183 and PapMV CP Loop6-C. Cloning, expression in E. coli, purification, SDS-PAGE, isolation of VLPs and DLS analysis of the recombinant proteins was conducted as described below.

The loop 6 peptide was fused at the different positions in the PapMV CP gene using PCR and the oligonucleotides showed in Table 3, below. In brief, a plasmid pET-3D containing the nucleotide sequence encoding the CP variant “PapMV CPsm” was used as a PCR template. PapMV CPsm harbours a deletion of the five N-terminal amino acids and includes a 6×His tag at the C-terminus. A multiple cloning site is included between the 6×His tag and the C-terminus to include SpeI and MluI restriction sites resulting in the addition of five amino acids (TSTTR) at this position (see FIG. 1D; SEQ ID NO:3).

Each of the primer combinations showed in Table 3 was used to introduce the fusion and generate a PCR product that contains the entire plasmid including the PapMV CPsm engineered protein. The PCR product is a linear dsDNA product that was further digested with the restriction enzyme Acc651 (New England Biolabs, Ipswich, Mass.) (underlined in Table 3). The restriction enzyme was inactivated by heat or by phenol/chloroform extraction. The resulting digested DNA was self-ligated using T4 DNA ligase. The ligated product resulted in a fully competent plasmid containing the newly engineered PapMV CP. The sequences were verified by DNA sequencing. The plasmid was used to transform E. coli strain BL21 for expression and purification of the proteins.

TABLE 3
Oligonucleotide Sequences

| | | | SEQ |
|---|---|---|---|
| | | | ID |
| Name | | Oligonucleotide Sequence | NO |
| Loop6-8 | Forward | 5′-ACGTGGTACCTCTAACGGTTCTAACC | 30 |
| | | CGTCTACCTCTTACGGTTTCGCGAACTTC | |
| | | CCCGCCATCACCCAGGAACAAATG-3′ | |
| | Reverse | 5′-ACGTGGTACCGGCTATGTTGGGTGTG | 31 |
| | | GATGC-3′ | |
| Loop6-183 | Forward | 5′-ACGTGGTACCTCTAACGGTTCTAACC | 32 |
| | | CGTCTACCTCTTACGGTTTCGCGAACAAC | |
| | | AACTTTGCCAGCAACTCCGCCTTC-3′ | |
| | Reverse | 5′-ACGTGGTACCGTCCTGTGCCGCGGCT | 33 |
| | | TGGAA-3′ | |
| Loop6-C | Forward | 5′-CTAGTGGTACTTCTAACGGTTCTAAC | 34 |
| | | CCGTCTACTTCTTACGGTTTCGCGA | |
| | | ACA-3′ | |
| | Reverse | 5′-CTAGTGTTCGCGAAACCGTAAGAAGT | 35 |
| | | AGACGGGTTAGAACCGTTAGAAGTA | |
| | | CCA-3′ | |

Expression and purification of PapMV constructs were performed as previously described with minor modifications (Tremblay et al., 2006, FEBS, 273:14-25). Briefly, the bacteria were lysed through a French press and then loaded onto a Ni²⁺ column, washed with 10 mM Tris-HCl/50 mM Imidazole/0.5% Triton X100 (pH 8), then with 10 mM Tris-HCl/50 mM Imidazole/1% Zwittergent (pH 8) to remove endotoxin contamination. Following elution of the proteins, the solutions were dialyzed against Tris-HCl 10 mM pH 8, using a 6-8 kDa molecular weight cut-off membrane (Spectra) for 12-16 hours. The dialysed proteins were subjected to high speed centrifugation (100,000×g) for 45 min in a Beckman 50.2 TI rotor. The VLP pellet was resuspended in endotoxin-free PBS (Sigma-Aldrich). Protein solutions were filtered using 0.22-0.45 μM filters before use. The purity of the proteins was determined by SDS-PAGE. The amount of protein was evaluated using a BCA protein kit (Pierce). Levels of expression for each recombinant protein were determined by SDS-PAGE. LPS contamination in the purified protein was evaluated with the Limulus test according to the manufacturer's instructions (Cambrex) and was less than 5 EU/mg of recombinant proteins.

The size and structure of the VLPs comprising the loop 6 fusions were confirmed by observation on a TEM (JEOL-1010, Tokyo, Japan). Dynamic light scattering (DLS) was also used to determine the average size of the VLPs.

### Results

The results are shown in FIG. 9. FIG. 9B depicts SDS-PAGE analysis of the recombinant PapMV CP fusion proteins, where Lane 1 contains bacterial lysate of the bacteria before induction; Lane 2 contains bacterial lysate of the bacteria after expression of the protein PapMV Loop6-8; Lane 3 contains purified PapMV Loop6-8; Lane 4 contains bacterial lysate of the bacteria before induction; Lane 5 contains bacterial lysate of the bacteria after expression of the protein PapMV Loop6-183; Lane 6 contains purified PapMV Loop6-183; Lane 7 contains bacterial lysate of the bacteria before induction; Lane 8 contains bacterial lysate of the bacteria after expression of the protein PapMV Loop6-C; and Lane 9 contains purified PapMV Loop6-C. In all cases, the recombinant proteins were well expressed in E. coli and were easily purified by affinity chromatography on a Ni²⁺ column.

FIG. 9C shows electron micrographs of the VLPs comprising PapMV Loop6-8, PapMV Loop6-183 and PapMV Loop6-C, respectively. FIG. 9D shows the results of dynamic light scattering (DLS) of the VLPs comprising PapMV Loop6-8, PapMV Loop6-183 and PapMV Loop6-C, which confirmed the average size of the VLPs to be approximately 80 nm for all the constructs harboring a fusion the loop 6 epitope.

### Example 3

### Ability of VLPs Comprising Recombinant PapMV CP-Loop 6 Peptide Fusions to Elicit a Humoral Immune Response

The following experiment was performed to assess the ability of PapMV VLPs comprising the CP fusion proteins described in Example 2 to elicit a humoral response in mice.

Briefly, five mice per group were immunized with 100 μg of each of the different VLPs described in Example 2, except for the group immunized with PapMV Loop6-C VLPs, which included 4 mice.

On day 28, the mice were bled and the immune response assessed by standard ELISA using a GST protein fused to the loop 6 synthetic peptide. ELISAs were performed against the PapMV VLPs (0.1 μg/mL) to evaluate the anti-PapMV response and against the loop 6 peptide (0.1 μg/mL) to evaluate the anti-loop 6 response. The general procedure described in Denis et al. (2008, Vaccine, 26; 3395-3403) was followed.

### Results

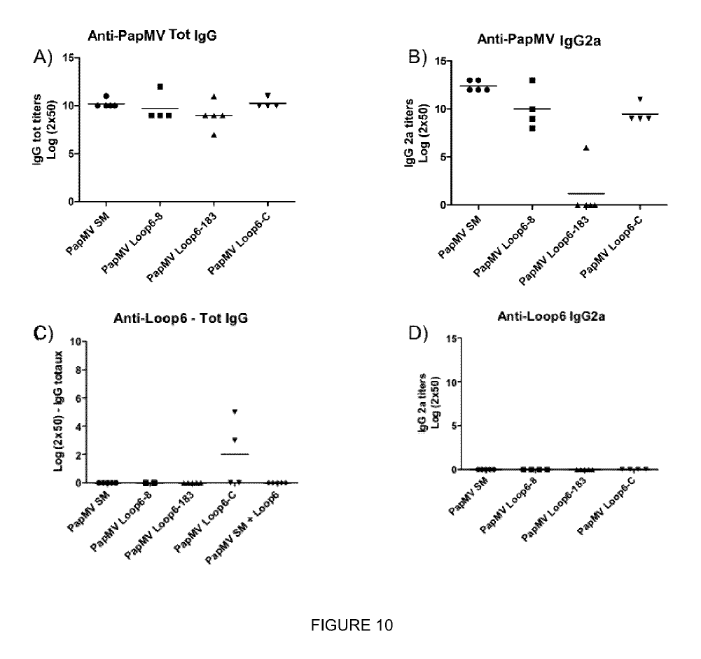

The results are shown in FIG. 10 and confirm that the PapMV platform harboring loop-6 fusion is highly immunogenic. Production of PapMV specific total IgG was triggered when animals were immunized with VLPs comprising any of PapMV CPsm, PapMV CP Loop6-8, PapMV CP Loop6-183 and PapMV CP Loop6-C (FIG. 10A), although the IgG2a titers directed to the platform were very low for the PapMV CP Loop6-183 construct (FIG. 10B). Only the PapMV Loop6-C VLPs induced a detectable total IgG response toward the loop-6 peptide (FIG. 10C). None of the VLPs triggered a detectable IgG2a response toward the loop-6 peptide (FIG. 10D). As all the VLPs were highly immunogenic, it is likely that the fusion of the loop 6 peptide to the CP may affect the structure of the peptide such that antibodies raised to the fused peptide do not react with the free peptide (as used in the ELISA). The fusion at the C-terminus, however, appeared not to affect the structure of the peptide as much as IgG from some of the mice immunized with the PapMV Loop6-C VLPs was able to bind free peptide in the ELISA. As not all the mice immunized with the PapMV Loop6-C VLPs could mount an immune response, it is likely that the immune repertoire of the mice differs from one individual to another, which is an effect often observed in such experiments.

### Example 4

### Preparation of VLPs Comprising Recombinant PapMV Coat Proteins Fused to a CTL Epitope

The following experiment describes the preparation of recombinant PapMV CPs fused to a CTL epitope derived from the highly conserved NP protein of the virus influenza. This peptide (TYQRTRALV [SEQ ID NO:36] also referred to as NP₁₄₇₋₁₅₅) is an H-2d CTL epitope of Balb/c that can be used to induce a protective CTL response to infection with influenza in a mouse model (Fu et al., 1997, J. Virol., 71:2715-2721; Tao et al., 2009, Antiviral Research, 81:253-160). Therefore, this peptide was chosen to evaluate the capacity of the PapMV VLPs to induce an IFN-γ cellular response in the Balb/c murine model.

Proteins harboring the fusion of the NP CTL peptide at position 8, position 183 or at the C-terminus of PapMV CP were produced (PapMV NP-8, PapMV NP-183 and PapMV NP-C, respectively). The NP peptide was fused at the different positions in the PapMV CP gene using PCR and the oligonucleotides shown in Table 4, below. The protocols outlined in Examples 2 and 3 were used for cloning, expression in E. coli, SDS-PAGE analysis, purification and production of VLPs. Discs were separated from the VLPs by high speed ultracentrifugation (as described in Denis et al., 2007, Virology, 363:59-68, and Denis et al., 2008, Vaccine, 26; 3395-3403).

TABLE 4
Oligonucleotide Sequences

| | | | SEQ |
|---|---|---|---|
| | | | ID |
| Name | | Oligonucleotide Sequence | NO |
| PapMV | Forward | 5′-AGCTCGTACGCGTGCGCTGGTTC | 37 |
| NP-8 | | GTACCGGTATGGACTTCCCCGCCATC | |
| | | ACCCAGGAAC-3′ | |
| | Reverse | 5′-TCGACGTACGCTGGTAGGTCGCG | 38 |
| | | TCGTTCAGGTTGGCTATGTTGGGTGT | |
| | | GGATGCC-3′ | |
| PapMV | Forward | 5′-AGCTCGTACGCGTGCGCTGGTTC | 39 |
| NP-183 | | GTACCGGTATGGACAACAACTTTGCC | |
| | | AGCAACTCCGCC-3′ | |
| | Reverse | 5′-TCGACGTACGCTGGTAGGTCGCG | 40 |
| | | TCGTTCAGGTTGTCCTGTGCCGCGGC | |
| | | TTGGAAGAG-3′ | |
| PapMV | Forward | 5′-ACGTCGTACGCGTGCGCTGGTTC | 41 |
| NP-C | | GTACCGGTATGGACACGCGTCACCAT | |
| | | CACCATCAC-3′ | |
| | Reverse | 5′-TCGACGTACGCTGGTAGGTCGCG | 42 |
| | | TCGTTCAGGTTACTAGTTTCGGGGGG-3′ | |

### Results

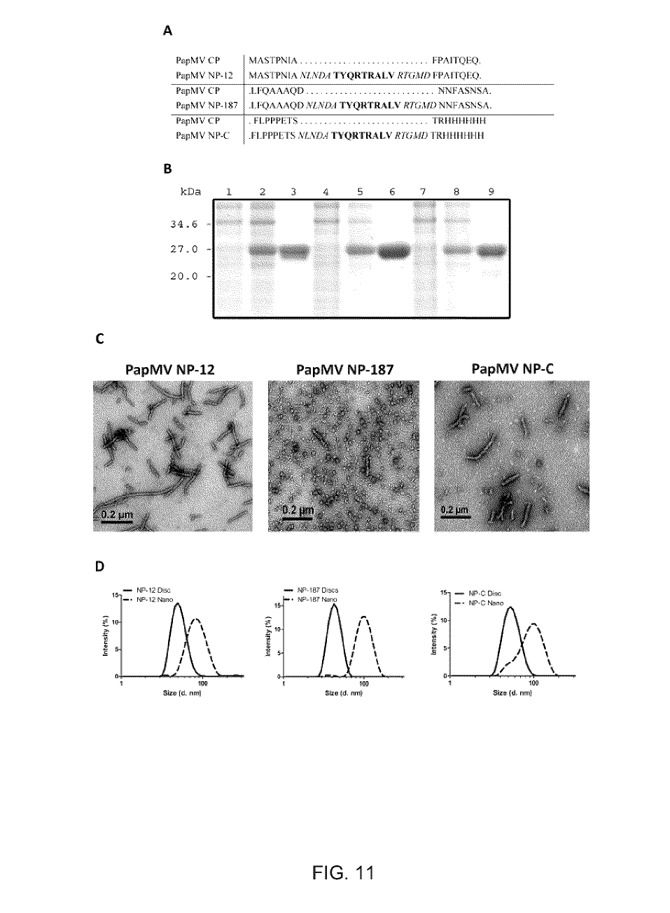

The results are shown in FIG. 11. Three different recombinant PapMV CP fusions with the CTL epitope inserted after amino acid 8, 183 and at the C-terminus of the protein were generated (FIG. 11A). On each side of the CTL epitope, 5 flanking amino acids (NLNDA [SEQ ID NO:43] and RTGMD [SEQ ID NO:44]) were added to ensure adequate processing of the CTL epitope in mouse antigen presenting cells (APCs) as shown in FIG. 11A.

FIG. 11B shows SDS-PAGE analysis of the fusion proteins where the lanes contain the following: Lane 1: bacterial lysate of the bacteria before induction; Lane 2: Bacterial lysate of the bacteria after expression of the protein PapMV NP-8: Lane 3: purified PapMV NP-8: Lane 4: bacterial lysate of the bacteria before induction: Lane 5: bacterial lysate of the bacteria after expression of the protein PapMV NP-183: Lane 6: purified PapMV NP-183: Lane 7: bacterial lysate of the bacteria before induction; Lane 8: bacterial lysate of the bacteria after expression of the protein PapMV NP-C, and Lane 9: purified PapMV NP-C. High levels of expression were observed for the three constructs, and all of the engineered PapMV fusions were able to form VLPs (FIG. 11C). The size of the discs and the VLPs formed by each fusion protein was evaluated by DLS (FIG. 11D). All the VLPs showed an expected average length of approximately 90 nm (for example, 80 nm for PapMV NP-8 and 88 nm for PapMV NP-C). The discs showed an average diameter of approximately 30 nm with all the constructs (for example, 28 nm for PapMV NP-8 and 32 nm for PapMV NP-C).

### Example 5

### Ability of VLPs Comprising Recombinant PapMV CP-NP Fusions to Elicit a CTL Immune Response

Immunization Schedule with PapMV-NP Constructs

Five 6-8-week-old BALB/c mice (Charles River, Wilmington, Mass.) were immunized intraperitoneally (i.p.) three times at 2-week intervals with 100 μg of recombinant PapMV CPsm, PapMV NP-8, PapMV-NP-183 and PapMV NP-C. Mice were immunized with either VLPs or discs harbouring the same fusion. Two weeks after the last boost, the mice were sacrificed, the mice spleens were removed and splenocytes isolated as described below.

### ELISPOT and Secretion of IFN-γ

The day before splenocyte isolation, ethanol (70%) treated MultiScreen-IP opaque 96-well plates (High Protein Binding Immobilon-P membrane, Millipore, Bedford, Mass.) were coated overnight at 4° C. with 100 μl/well of capture IFN-γ antibody, diluted in DPBS (Abcam, Cambridge, Mass., USA) as suggested by the manufacturer in the murine interferon-gamma ELISPOT kit (Abcam, Cambridge, Mass., USA). After overnight incubation, the plates were washed three times with 200 μl PBS/well and blocked with 100 μl/well of 2% skimmed dry milk in PBS for 2 h at 37° C., 5% CO₂.

Two weeks after the last boost, the mice were sacrificed and the mouse spleens were removed aseptically. Spleens were minced in culture medium and homogenates were passed through a 100-μm cell strainer. The cells were centrifuged and red blood cells were removed by 5 min. room temperature incubation in ammonium chloride-potassium lysis buffer (150 mM NH₄Cl, 10 mM KHCO₃, 0.1 mM Na₂EDTA (pH 7.2-7.4)). Isolated red blood cell-depleted spleen cells were washed twice in PBS and diluted in culture media (RPMI 1640 supplemented with 25 mM HEPES, 2 mM L-glutamine, 1 mM sodium pyruvate, 1 mM 2-mercaptoethanol, 10% heat inactivated fetal bovine serum, 100 U/ml penicillin and 100 μg/ml streptomycin (Invitrogen, Canada). Duplicates at 2.5×10⁵ cells/well were reactivated with either culture medium alone or with NP₁₄₇₋₁₅₅ peptide (5 μg/ml) and were cultured for 36 h at 37° C. with 5% CO₂. At the end of incubation, the plates were washed manually 3 times with 200 μl/well of PBS/0.1% Tween 20. Biotinylated detection anti-mouse IFN-γ antibody in PBS/1% BSA was added at 100 μl/well and the plates were incubated for 90 min at 37° C., 5% CO₂. Plates were manually washed 3 times with PBS and 100 μl/well of streptavidin-alkaline phosphatase conjugated secondary antibody diluted in PBS/1% BSA was added for 1 h at 37° C., 5% CO₂. The plates were washed a final 3 times with PBS/0.1 Tween 20. Spots were visualized by adding 100 μl of ready-to-use BCIP/NBT buffer in each well for 2-15 min. The spots were counted under a binocular microscope. The precursor frequency of specific T cells was determined by subtracting the background spots in media alone from the number of spots seen in wells reactivated with the peptide.

### Results

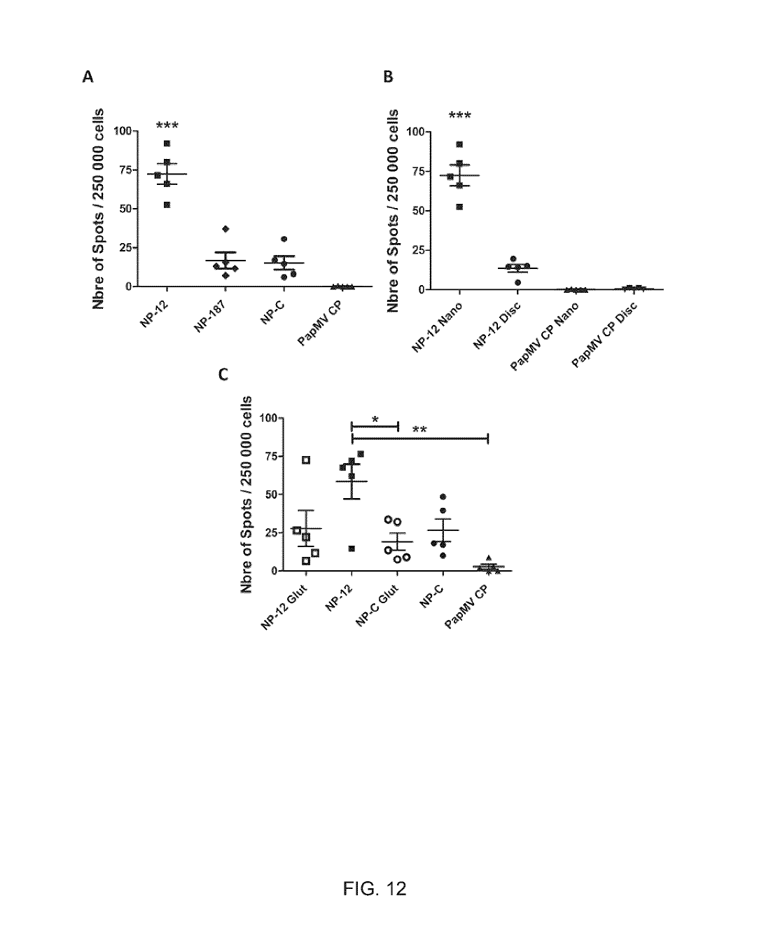

Since the level of IFN-γ secreted by purified splenocytes will be proportional to the level of precursors of CD8+ cytotoxic lymphocytes specific to the fused peptide, assessment of IFN-γ secretion allows the ability of each recombinant VLP to induce a cellular immune response in BALB/c mice to be compared. The results are shown in FIG. 12A and indicate that, although all the PapMV VLPs fused to the NP₁₄₇₋₁₅₅ peptide were able to induce secretion of IFN-γ at a level higher than PapMV CP VLPs alone, only the PapMV NP-12 VLPs triggered secretion of IFN-γ that was significantly greater than PapMV CP VLPs alone. Surprisingly, the amount of IFN-γ secretion induced by PapMV NP-C VLPs was not significantly different from that induced by PapMV CP VLPs alone even though PapMV NP-C were well presented to the immune system, as evidenced by production of antibody directed to the vaccine platform after three immunisations with PapMV NP-C VLPs.

As shown in FIG. 12B, the level of IFN-γ secreted by splenocytes specific to PapMV NP-12 VLPs was significantly higher than the level triggered by PapMV NP-12 discs indicating that self-assembly into VLPs is required for strong stimulation of the immune system, both in terms of humoral and cellular response. The ability of the discs to stimulate a low response, however, suggests that preparations of VLPs that comprise low amounts of discs could still be used for effective stimulation of an immune response.

### Example 6

### Stability of VLPs Comprising Recombinant PapMV CP-NP Fusions

### Dynamic Light Scattering

For dynamic light scattering (DLS), the size of the VLPs was recorded with a ZetaSizer Nano ZS (Malvern, Worcestershire, United Kingdom) at a temperature of 10° C. at a concentration of 0.1 mg/ml diluted in PBS 1×. The variation in VLP size induced by temperature variations was measured at temperature increments of 1° C. according to the same experimental conditions.

Chemical Cross-Linking with Glutaraldehyde

0.1% glutaraldehyde in 10 mM Tris, 50 mM NaCl pH 7.5 in a final volume of 50 μl was used. The optimal concentration of protein used to cross-link was 150 ng/ml. After addition of glutaraldehyde, the mixture was incubated at room temperature for 30 minutes in the dark. The reaction was stopped with 15 μl of loading dye and heated 10 minutes at 95° C. and the proteins were separated by SDS-PAGE. The cross-linked proteins used for immunization were stored at 4° C. until immunization without adding loading dye.

### Trypsin Digestion

10 μg of protein was incubated at 37° C. in a volume of 50 μl for 120 minutes in 100 mM Tris-HCl pH 8.5 with 0.2 μg trypsin (Roche, 1418475). The reaction was stopped by adding 10 μl of loading dye. Samples were heated 10 minutes at 95° C. prior to analysis by SDS-PAGE.

### Results

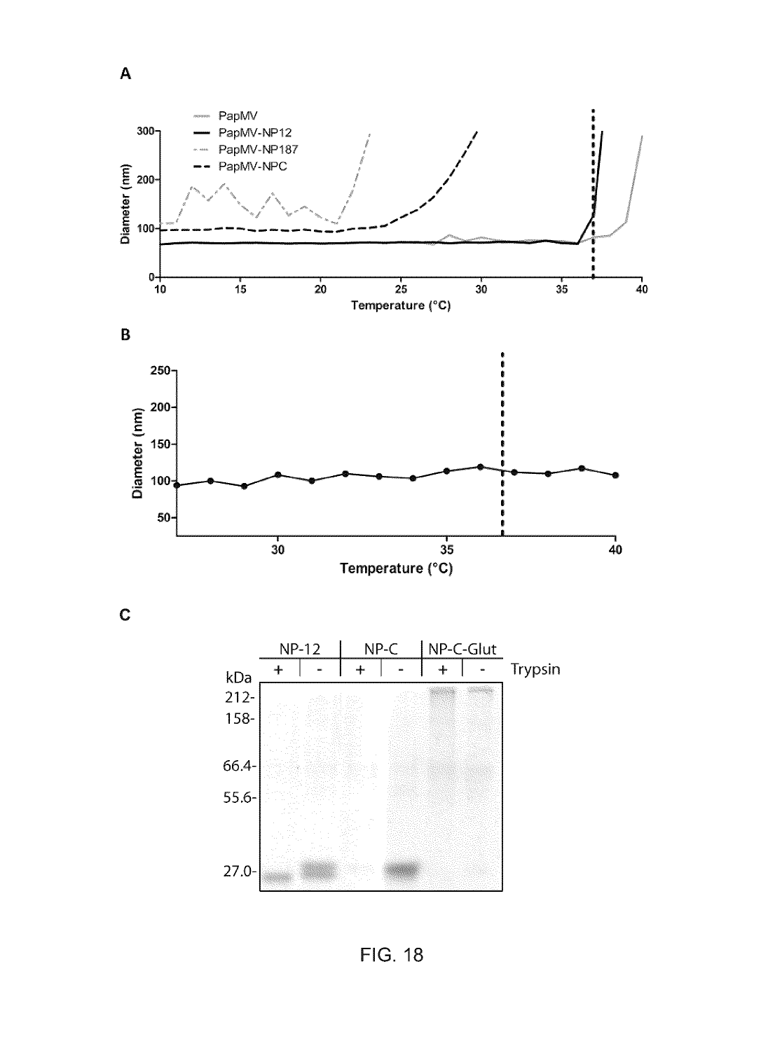

DLS was used to assess the stability of PapMV NP-12 PapMV NP-187, PapMV NP-C and PapMV CP VLPs (FIG. 18A). Both PapMV NP-187 VLPs and PapMV NP-C VLPs started to aggregate at temperatures lower than mouse body temperature (36.9° C.) (approximately 20° C. and 25° C., respectively). In contrast, PapMV NP-12 VLPs started to aggregate at a temperature around 37° C. which is similar to PapMV CP VLPs (FIG. 18A). This greater stability of the PapMV NP-12 VLPs correlates well with their ability to stimulate a CTL response as shown in Example 5.

Cross-linking of the recombinant PapMV NP-C VLPs using glutaraldehyde was investigated as a way to stabilise the VLPs and potentially increase their ability to stimulate an immune response. DLS was used to assess the stability of the cross-linked PapMV NP-C VLPs and showed that the VLPs were stable at 37° C. (FIG. 18B).

The cross-linked PapMV NP-C VLPs (100 μg) were also used to immunize mice, with the non-cross linked PapMV NP-C VLPs and PapMV CP VLPs (100 μg of each) as comparators. The level of IFN-gamma secreted by specific splenocytes was measured as described in Example 5. Cross-linked PapMV NP-C VLPs did not induce a high level of IFN-gamma after stimulation of the splenocytes with NP₁₄₇₋₁₅₅ peptide (FIG. 12C), with the quantity of IFN-gamma secreted by specific splenocytes remaining similar to that obtained with the un-crosslinked PapMV NP-C VLPs, demonstrating that stability at physiological temperature alone is not sufficient to confer on this fusion the ability to stimulate an efficient cellular response.

Trypsin digestion of PapMV CP-NP fusions was used to investigate the possibility that cross-linking may be inhibiting cleavage of the NP₁₄₇₋₁₅₅ peptide by cellular proteases. As shown in FIG. 18C, no difference was observed between untreated cross-linked PapMV NP-C VLPs and cross-linked PapMV NP-C VLPs treated with trypsin (both bands can be seen to have the same intensity). This is in contrast to PapMV NP-12 and PapMV NP-C VLPs which were well digested by trypsin (see FIG. 18C).

These results indicate that the cross-linking of PapMV VLPs results in access to the NP₁₄₇₋₁₅₅ peptide by cellular proteases being sterically hindered, either by masking the peptide and/or by increasing the rigidity of the VLPs such that protease cleavage cannot occur.

### Example 7

### Preparation of Recombinant PapMV Coat Proteins Fused to Multiple Copies of an Influenza Nucleocapsid Peptide

This experiment describes the preparation and analysis of recombinant PapMV coat protein harbouring 2 or 3 CTL peptides inserted at a single position in the CP or at different positions in the CP. The NP₁₄₇₋₁₅₅ peptide described in Example 4 was used in these experiments.

The constructs produced were:
PapMV 3NP-C—with 3 copies of the NP CTL peptide inserted at the C-terminus;
PapMV NP-8/183—with one NP CTL peptide inserted after amino acid 8 and one inserted after amino acid 183;
PapMV NP-8/C—with one NP CTL peptide inserted after amino acid 8 and one inserted at the C-terminus;
PapMV NP-183/C—with one NP CTL peptide inserted after amino acid 183 and one at the C-terminus;
PapMV 3NP-8—with 3 copies of the NP CTL peptide inserted after amino acid 8, and
PapMV 3NP-8/183/C (PapMV triple NP)—with one NP CTL peptide inserted after amino acid 8, one inserted after amino acid 183, and one inserted at the C-terminus.

The protocols outlined in Examples 2 and 3 were used for cloning, expression in E. coli, SDS-PAGE analysis, purification and VLP preparation. Discs were separated from the VLPs by high speed ultracentrifugation (Denis et al., 2007, 2008, ibid.).

### Results

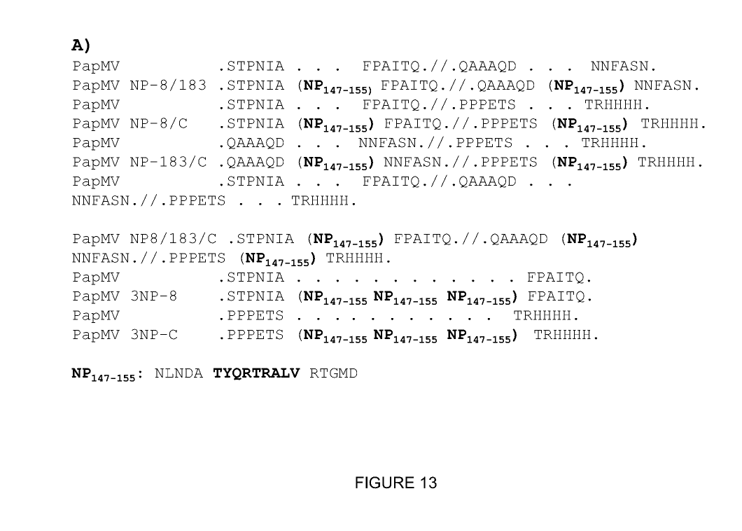

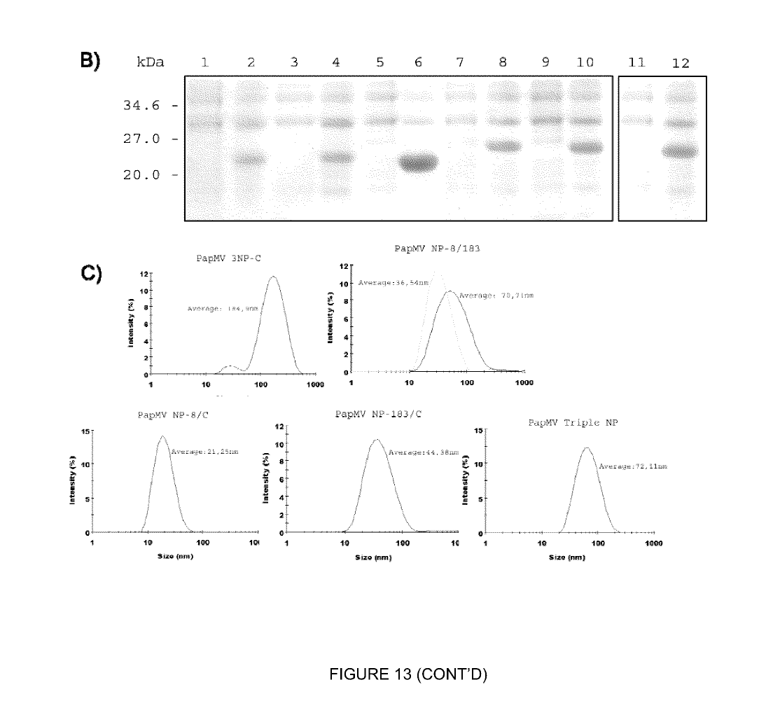

Amino acid sequences at the site(s) of insertion are shown in FIG. 13A. FIG. 13B depicts SDS-PAGE analysis of the expression of these recombinant PapMV CP fusions: Lane 1: Bacterial lysate of the bacteria before induction; Lane 2: Bacterial lysate of the bacteria after expression of the multifusion protein PapMV-NP8/183; Lane 3: Bacterial lysate of the bacteria before induction; Lane 4: Bacterial lysate of the bacteria after expression of the multifusion protein PapMV-NP8/C; Lane 5: Bacterial lysate of the bacteria before induction; Lane 6: Bacterial lysate of the bacteria after expression of the protein PapMV-NP183/C; Lane 7: Bacterial lysate of the bacteria before induction; Lane 8: Bacterial lysate of the bacteria after expression of the multifusion protein PapMV-triple NP; Lane 9: Bacterial lysate of the bacteria before induction; Lane 10: Bacterial lysate of the bacteria after expression of the multifusion protein PapMV-3NP/8; Lane 11: Bacterial lysate of the bacteria before induction, and Lane 12. Bacterial lysate of the bacteria after expression of the multifusion protein PapMV-3NP/C.

FIG. 13C depicts dynamic light scattering (DLS) analysis of the PapMV VLPs 3NP-C, NP-8/183, NP-8/C, NP-8/C and triple NP. The average length of the VLPs is indicated on each graph. It is considered that the PapMV CP forms a VLP only when the length exceeds 40 nm as measured by DLS.

FIGS. 13B and C indicate that all constructs were able to produce a stable protein in E. coli and to self-assemble into VLPs. The VLPs produced by these constructs can be used to immunize mice and evaluate their ability to improve the immune response to the NP peptide as compared to PapMV VLPs that harbor only one fusion of the same peptide.

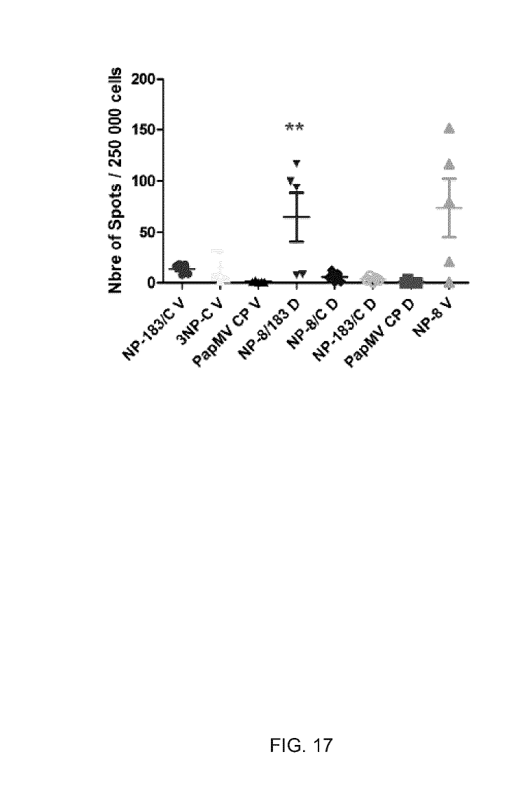

FIG. 17 shows the results of an ELISPOT analysis (performed essentially as described in Example 3) of VLPs (V) and discs (D) of the various constructs.

### Example 8

### Peptide Mapping of Surface-Exposed Regions of the PapMV Coat Protein

### Peptide Synthesis

The entire amino acid sequence of PapMV-CP was synthesised in short peptides by GenScript (Piscataway, N.J., USA) and these were used as crude peptides without HPLC purification. The peptides were designed to be 12 amino acids in length and each one overlapped with flanking peptides by 4 amino acids (Table 5). The cysteines in peptides 8 and 13 were changed to serines to avoid the possible interference of sulphide bonds with other compounds in the experiments. The peptides containing cysteines were also tested and the results showed that these did not produce any interference peptides by GenScript (Piscataway, N.J., USA) and were used as crude, without been HPLC purified. Peptides were 12 amino acids long and overlap by 4 amino acids at each ends with the succeeding and preceding peptides (Table 5). Cysteines in peptide 8 and 13 were change for serines to avoid the possible interference of sulphide bonds with other compounds in the experiments. Peptides containing cysteines were also tested and showed not producing any interference.

### Immunization of PapMV VLPs and Immunodotblot

Five 6 to 8-week-old BALB/c mice were injected subcutaneously 199 with 100 μg of PapMV VLPs. A booster shot was given 2 weeks after the first injection and blood samples were obtained 2 weeks after the boost. Peptides were applied in duplicate onto Nexterion-E slides MPX 16 (Schott, Elmsford, N.Y., USA) following the manufacturer's protocol. Slides were then blocked for 1 hour at room temperature with PBS+Tween®20 0.05%+BSA 1%. Pooled sera from five immunized mice were placed in duplicate on the array at a dilution of 1:100 in blocking buffer for 1 hour at room temperature. The peptides antibodies were detected using Alexa-fluor 647 anti-mouse IgG goat antibodies (Invitrogen, Carlsbad, Calif., USA) at a dilution of 1:800 for 1 hour. Slides were washed three times between each step with PBS-T for 3 minutes at room temperature. Glass slides were read using ScanArray 4000XL (GSI Lumonics) and analysed with GenePix 6.1.0.4 (Molecular devices).

TABLE 5
PapMV CP Peptides

| Peptide | | SEQ |
|---|---|---|
| # | Sequence | ID NO |
| 1 | MASTPNIAFPAI | 45 |
| 2 | FPAITQEQMSSI | 46 |
| 3 | MSSIKVDPTSNL | 47 |
| 4 | TSNLLPSQEQLK | 48 |
| 5 | EQLKSVSTLMVA | 49 |
| 6 | LMVAAKVPAASV | 50 |
| 7 | AASVTTVALELV | 51 |
| 8 | LELVNFSYDNGS | 52 |
| 9 | DNGSSAYTTVTG | 53 |
| 10 | TVTGPSSIPEIS | 54 |
| 11 | PEISLAQLASIV | 55 |
| 12 | ASIVKASGTSLR | 56 |
| 13 | TSLRKFSRYFAP | 57 |
| 14 | YFAPIIWNLRTD | 58 |
| 15 | LRTDKMAPANWE | 59 |
| 16 | ANWEASGYKPSA | 60 |
| 17 | KPSAKFAAFDFF | 61 |
| 18 | FDFFDGVENPAA | 62 |
| 19 | NPAAMQPPSGLT | 63 |
| 20 | SGLTRSPTQEER | 64 |
| 21 | QEERIANATNKQ | 65 |
| 22 | TNKQVHLFQAAA | 66 |
| 23 | QAAAQDNNFASN | 67 |
| 24 | FASNSAFITKGQ | 68 |
| 25 | TKGQISGSTPTI | 69 |
| 26 | TPTIQFLPPPE | 70 |
| 27 | PPETSTTR | 71 |

### Results

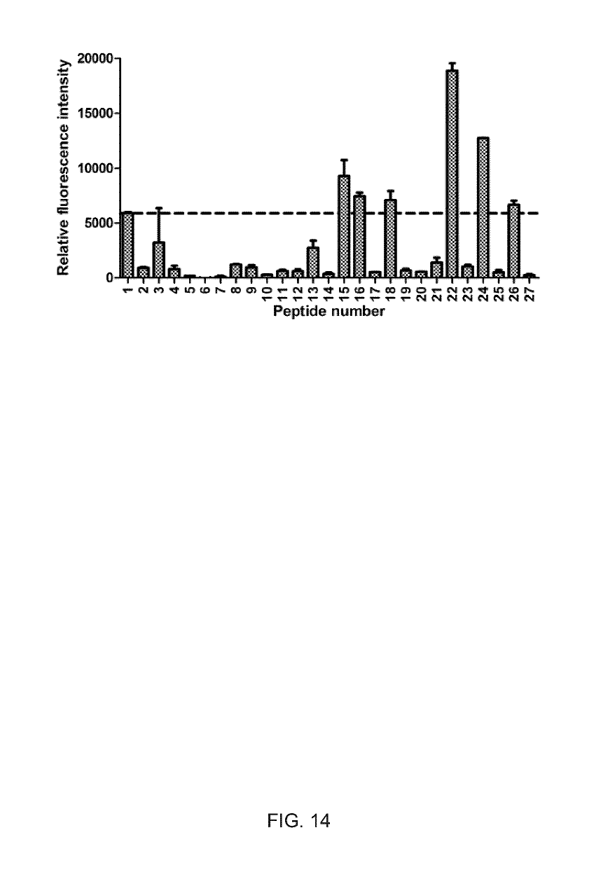

FIG. 14 shows the results from the immunodot analysis. As expected, peptides corresponding to the N- and C-termini were detected by the polyclonal antibodies. In addition, PapMV polyclonal antibodies could also detect (with a high affinity) five other regions corresponding to peptides 15, 16, 18, 22 and 24. The same experiment was performed using individual serum from a single mouse and essentially the same results were obtained, but with a variation in the intensity of the signal registered for peptides 18, 22 and 24. However, consistent in all mice, peptides 15 and 16 give a strong signal.

### Example 9

### Analysis of Chemically Modified Surface-Exposed Residues of the PapMV Coat Protein

Chemical Modifications with DEPC and EDC

PapMV nanoparticles were chemically modified in solution with chemically active compounds that interact selectively with certain amino acids; carboxyl groups with 1-ethyl-3-(3-dimethylaminopropyl)carbodiimide (EDC); and serine, threonine, histidine and tyrosine with diethylpyrocarbonate (DEPC). Both reactions were carried out with 1 mg/ml of PapMV VLPs in a volume of 100 μl. Briefly, the EDC reaction was performed by adding EDC to obtain a concentration of 2.0 mM in 50 mM glycinamide hydrochloride buffer pH 6.0 and by incubating this reaction at room temperature for 1 hour. The DEPC reaction was performed with a concentration of 0.4 mM DEPC in 50 mM ammonium acetate+1% acetonitrile solution for 1 minute at 37° C. VLPs were washed by two centrifugations at 14 000×g for 15 minutes in an Amicon Ultra 10 kDa MWCO 0.5 ml (Millipore, Billerica, Mass., USA) with ammonium acetate 50 mM for DEPC and Tris-HCl 10 mM pH 8.0 for EDC. The integrity of the VLPs was verified by electron microscopy and dynamic light scattering. The digest and mass spectrometry experiments were performed by the Proteomics platform of the Eastern Quebec Genomics Center, Quebec, Canada.

### Electron Microscopy and Dynamic Light Scattering

VLPs were diluted in water to a concentration of 0.03 mg/ml and stained by mixing 10 μl of sample with 10 μl of 3% acetate-uranyl for 7 minutes in the dark before putting 8 μl of this solution on carbon-formvar grids for 5 minutes. Grids were observed with a JEOL-1010 transmission electron microscope (Tokyo, Japan). The size of the VLPs was recorded with a ZetaSizer Nano ZS (Malvern, Worcestershire, United Kingdom) at a temperature of 4° C. at a concentration of 0.1 mg/ml diluted in PBS 1×.

### Protein Digestion by Trypsin

Tryptic digestion was performed on a MassPrep liquid handling robot (Waters, Milford, USA) according to the manufacturer's specifications and the protocol of Shevchenko et al. (1996, Anal Chem 68:850-858) with the modifications suggested by Havlis et al. (2003, Anal Chem 75:1300-1306). Briefly, proteins were reduced with 10 mM DTT and alkylated with 55 mM iodoacetamide. Trypsin digestion was performed using 105 mM of modified porcine trypsin (Sequencing grade, Promega, Madison, Wis.) at 58° C. for 1 h. Digestion products were extracted using 1% formic acid, 2% acetonitrile followed by 1% formic acid, 50% acetonitrile. The recovered extracts were pooled, vacuum centrifuge dried and then resuspended in 7 μl of 0.1% formic acid; 2 μl were analyzed by mass spectrometry.

### Mass Spectrometry of the Modified VLPs

Peptide samples were separated by online reversed-phase (RP) nanoscale capillary liquid chromatography (nanoLC) and analyzed by electrospray mass spectrometry (ES MS/MS). The experiments were performed with a Thermo Surveyor MS pump connected to a LTQ linear ion trap mass spectrometer (ThermoFisher, San Jose, Calif. USA) equipped with a nanoelectrospray ion source (ThermoFisher, San Jose, Calif. USA). Peptide separation took place on a self packed PicoFrit column (New Objective, Woburn, Mass.) packed with Jupiter (Phenomenex) 5u, 300A C18, 10 cm×0.075 mm internal diameter. Peptides were eluted with a linear gradient from 2-50% solvent B (acetonitrile, 0.1% formic acid) for 30 minutes, at 200 mL/min (obtained by flow-splitting). Mass spectra were acquired using a data dependent acquisition mode using Xcalibur software version 2.0. Each full scan mass spectrum (400 to 2000 m/z) was followed by collision-induced dissociation of the seven most intense ions. The dynamic exclusion (30 seconds exclusion duration) function was enabled, and the relative collisional fragmentation energy was set to 35%.

### Database Searching

All MS/MS samples were analyzed using Mascot (Matrix Science, 265 London, UK; version 3.1.2). Mascot was set up to search the PapMV-CP amino acid sequence assuming the digestion enzyme trypsin. Mascot was searched with a fragment ion mass tolerance of 0.50 Da and a parent ion tolerance of 2.0 Da. The iodoacetamide derivative of cysteine was specified as a fixed modification, and oxidation of methionine was specified as a variable modification. Two missed cleavages were allowed.

### Criteria for Identification of Modifications

Scaffold (3.2.0, Proteome Software Inc., Portland, Oreg.) was used to validate MS/MS based peptide and protein identifications. Peptide identifications were accepted if they could be established at greater than 95.0% probability as specified by the Peptide Prophet algorithm (Keller et al., 2002, Anal Chem 74:5383-5392). Modifications were identified by a shift in the peptides mass of 56 Da for EDC and 72 Da for DEPC.

### Results

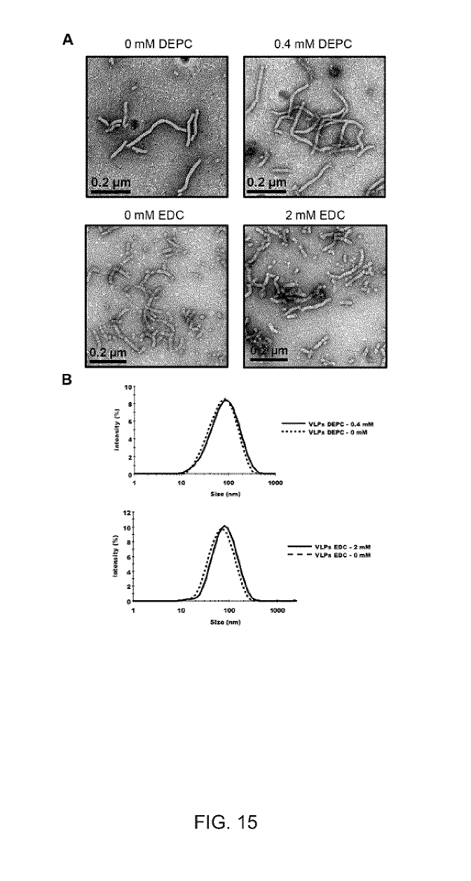

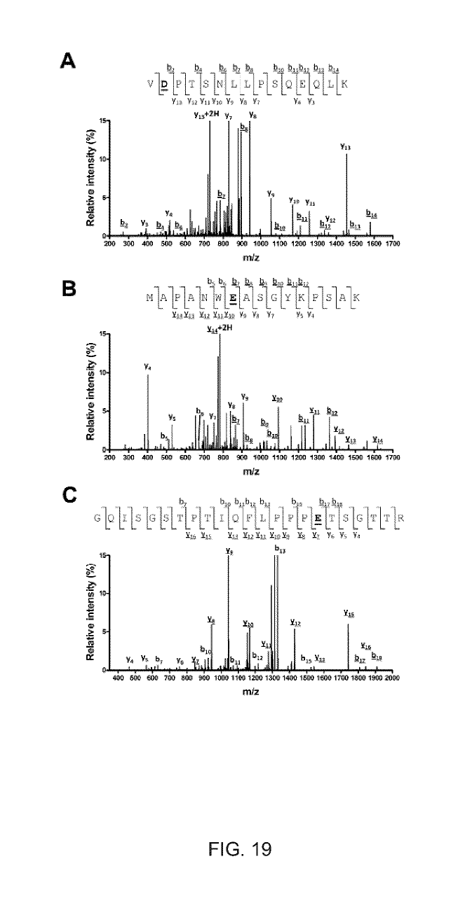

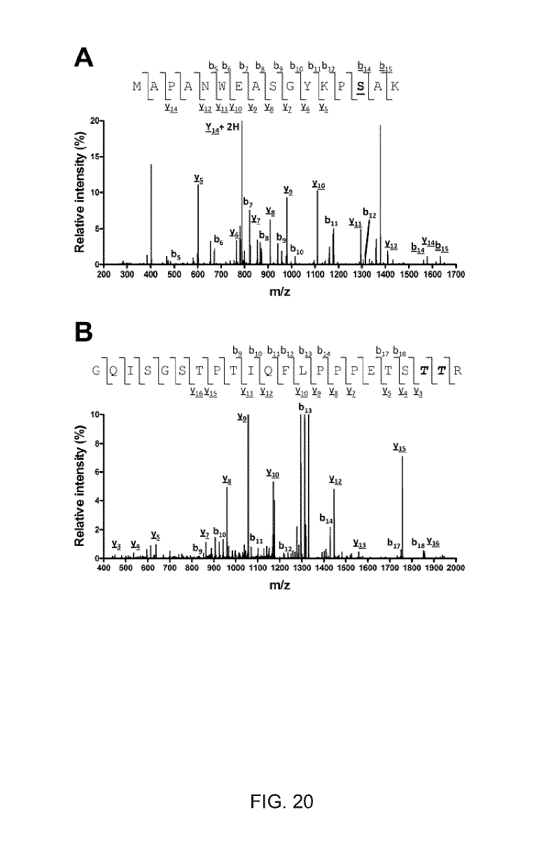

In order to confirm the immunological results described in Example 8, PapMV VLPs were modified chemically at surface-exposed residues and analyzed by mass spectrometry. The modified VLPs were also analyzed by electron microscopy and DLS to ensure that their general aspect and length were similar to those of untreated VLPs (FIG. 15). The VLPs were then digested with trypsin and analyzed by electrospray mass spectrometry. Approximately 70% of the amino acid sequence of PapMV coat protein could be analyzed for modifications after tryptic digestion. Modifications by EDC and DEPC add 56 Da and 72 Da to the molecular weight of the peptides, respectively. EDC modifications were found at position D17, E128 and E215 (FIG. 19) and DEPC modifications at S135 and T219 (FIG. 20) as shown on the MS/MS spectra. The N- and C-termini were also both chemically modified and were therefore confirmed to be located at the surface of PapMV VLPs. Interestingly, a central region, E128 and S135, appeared to be exposed at the surface of the VLPs as confirmed by immunoblot and MS/MS.

The immunoblot peptide array appeared to be more sensitive than MS/MS spectroscopy for mapping the surface of the VLPs. In fact, MS/MS can reveal only those modifications that predominate in the samples and are thus available for cross-linking. All the regions of PapMV VLPs exposed at the surface may not have been identified even with the combination of these two techniques, since the immunoblot technique can react only to linear epitopes presented by the array and MS/MS is limited by the efficiency of labeling of the surface through chemical cross-linking—the context has to be optimal to obtain good and sensitive resolution.

### Example 10

### Confirmation of Surface-Exposed Residues of the PapMV Coat Protein by Immunization of Mice

Peptide Coupling to mcKLH Adjuvant Proteins, Immunization and ELISA

Peptides were linked to mcKLH using the mcKLH linking kit (Pierce, Rockford, Ill., USA). Immunizations were performed using 100 μg of linked mcKLH with 10 μg of Quil-A saponin (Brenntag Biosector, Denmark) adjuvant for peptides 1, 13, 15, 16, 17, 18, 22, 24 and 26, with a 2-week interval before a boost shot. Sera of two mice per peptide were taken at day 28 to assay by native protein ELISA as described elsewhere (Savard et al., 2011, PLoS ONE 6:e21522) using native PapMV VLPs at 0.1 μg/ml as antigens. A titer was considered positive when the optical density was three-fold higher than that of the pre-immune serum.

### Results

To further confirm that the residues targeted by the antibodies and by chemical modifications (Examples 8 and 9) were on the surface of PapMV VLPs, antibodies against peptides 1, 15, 16, 18, 22, 24 and 26 were produced by fusion to mcKLH adjuvant protein. Fusions to peptides 13 and 17 were also produced as negative controls. All peptides expected to be at the surface were confirmed by high total IgG titers, except peptide 15 (Table 6). The two controls, peptides 13 and 17, were negative, as predicted.

TABLE 6
Antibodies Against Surface-Exposed Peptides

| | | Pre- | Recognized |
|---|---|---|---|
| Peptide # | | sent | by |
| and | Position | at the | Antibody |
| Sequence | in | Sur- | Against |
| [SEQ ID NO] | PapMV CP | face | Peptide |
| 1-MASTPNIAFPAI [45] | 5 to 16 | + | + |
| 13-TSLRKFCRYFAP [57] | 101 to 112 | − | − |
| 15-LRTDKMAPANWE [59] | 117 to 128 | + | − |
| 16-ANWEASGYKPSA [60] | 125 to 136 | + | + |
| 17-KPSAKFAAFDFF [61] | 133 to 144 | − | − |
| 18-FDFFDGVENPAA [62] | 141 to 152 | + | + |
| 22-TNKQVHLFQAAA [66] | 173 to 184 | + | + |
| 24-FASNSAFITKGQ [68] | 189 to 200 | + | + |
| 26-TPTIQFLPPPE [70] | 205 to 215 | + | + |

The disclosure of all patents, publications, including published patent applications, and database entries referenced in this specification are expressly incorporated by reference in their entirety to the same extent as if each such individual patent, publication, and database entry were expressly and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

The embodiments of the invention in which an exclusive property or privilege is claimed are defined as follows:

1. A fusion protein comprising a peptide antigen derived from influenza M2e peptide fused to a papaya mosaic virus (PapMV) coat protein after an amino acid that corresponds to amino acid 6, 7, 8, 9, 10, 11 or 12 of SEQ ID NO:1, wherein the fusion protein is capable of self-assembly to form a virus-like particle (VLP), and wherein the peptide antigen is 20 amino acids or less in length and comprises the general sequence: V-X1-T-X2-X3-X4-X5 [SEQ ID NO:96], wherein X1 is E or D; X2 is P or L; X3 is T or I; X4 is R or K, and X5 is N, S or K.

2. The fusion protein according to claim 1, wherein the peptide antigen is fused after an amino acid that corresponds to amino acid 6, 7 or 10 of SEQ ID NO:1.

3. The fusion protein according to claim 1, wherein the peptide antigen is fused after an amino acid that corresponds to amino acid 6 of SEQ ID NO:1.

4. The fusion protein according to claim 1, wherein the PapMV coat protein comprises an amino acid sequence as set forth in SEQ ID NO:4 and the peptide antigen is fused to the PapMV coat protein after amino acid 1, 2, 3, 4, 5, 6, 7 or 8 of SEQ ID NO:4.

5. The fusion protein according to claim 4, wherein the peptide antigen is fused to the PapMV coat protein after amino acid 2, 3 or 6 of SEQ ID NO:4.

6. The fusion protein according to any one of claims 1 to 5, wherein the peptide antigen is between about 7 and about 12 amino acids in length, or between about 7 and about 10 amino acids in length.

7. The fusion protein according to any one of claims 1 to 5, wherein the peptide antigen is between about 7 and about 9 amino acids in length.

8. The fusion protein according to any one of claims 1 to 8, wherein the peptide antigen comprises a sequence as set forth in any one of SEQ ID NOs:14-22 and 96-104.

9. The fusion protein according to any one of claims 1 to 8, wherein the peptide antigen comprises the sequence EVETPIRNE [SEQ ID NO:21] or VETPIRN [SEQ ID NO:22].

10. The fusion protein according to any one of claims 1 to 8, wherein the peptide antigen consists essentially of the sequence EVETPIRNE [SEQ ID NO:21] or VETPIRN [SEQ ID NO:22].

11. The fusion protein according to claim 4, wherein the fusion protein comprises an amino acid sequence as set forth in SEQ ID NO:23 from amino acid 1-224; in SEQ ID NO:24 from amino acid 1-222; in SEQ ID NO:25 from amino acid 1-221; in SEQ ID NO:26 from amino acid 1-219; in SEQ ID NO:27 from amino acid 1-224, or in SEQ ID NO:28 from amino acid 1-222.

12. The fusion protein according to claim 4, wherein the fusion protein comprises an amino acid sequence as set forth in SEQ ID NO:23 from amino acid 1-224.

13. The fusion protein according to any one of claims 1 to 12, wherein the VLP is stable at a temperature of at least 37° C.

14. A virus-like particle (VLP) comprising the fusion protein according to any one of claims 1 to 13.

15. A pharmaceutical composition comprising the VLP according to claim 14 and a pharmaceutically acceptable carrier.

16. The pharmaceutical composition according to claim 15, formulated as a vaccine.

17. A method of inducing an immune response against an influenza virus in a subject comprising administering to the subject an effective amount of the VLP according to claim 14.

18. A method of reducing the risk of a subject developing influenza comprising administering to the subject an effective amount of the VLP according to claim 14.

19. A method of immunizing a subject against infection with an influenza virus comprising administering to the subject an effective amount of the VLP according to claim 14.

20. The method according to any one of claims 17 to 19, wherein the VLP induces a humoral immune response in the subject.

21. A virus-like particle (VLP) comprising the fusion protein according to any one of claims 1 to 13 for use to induce an immune response against an influenza virus in a subject in need thereof.

22. Use of a virus-like particle (VLP) comprising the fusion protein according to any one of claims 1 to 13 to induce an immune response against an influenza virus in a subject in need thereof.

23. Use of a virus-like particle (VLP) comprising the fusion protein according to any one of claims 1 to 12 in the manufacture of a medicament for inducing an immune response against an influenza virus in a subject.

24. A virus-like particle (VLP) comprising the fusion protein according to any one of claims 1 to 13 for use to reduce the risk of a subject developing influenza.

25. Use of a virus-like particle (VLP) comprising the fusion protein according to any one of claims 1 to 13 to reduce the risk of a subject developing influenza.

26. Use of a virus-like particle (VLP) comprising the fusion protein according to any one of claims 1 to 13 in the manufacture of a medicament for reducing the risk of a subject developing influenza.

27. A virus-like particle (VLP) comprising the fusion protein according to any one of claims 1 to 13 for use to immunize a subject against infection with an influenza virus.

28. Use of a virus-like particle (VLP) comprising the fusion protein according to any one of claims 1 to 13 to immunize a subject against infection with an influenza virus.

29. Use of a virus-like particle (VLP) comprising the fusion protein according to any one of claims 1 to 13 in the manufacture of a medicament for immunizing a subject against infection with an influenza virus.

30. The VLP according to any one of claim 21, 24 or 27, or the use according to any one of claim 22, 23, 25, 26, 28 or 29, wherein the VLP induces a humoral immune response in the subject.

31. A pharmaceutical kit comprising the VLP according to claim 14 and instructions for use.


SEQUENCE LISTING (104 sequences)

SEQ ID NO: 1 - protein, 215 aa, Papaya Mosaic Virus
MSKSSMSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLIRSPTQEERIANATNKQVHLFQAAAQDNNFTSNSAFITKGQISGSTPTIQFLPPPE

SEQ ID NO: 2 - DNA, 648 bp, Papaya Mosaic Virus
atgtctaagtcaagtatgtccacacccaacatagccttccccgccatcacccaggaacagatgagctcgattaaggtcgatccaacgtccaatcttctgccctcccaagagcagttaaagtcagtgtccaccctcatggtagctgctaaggttccagcagccagtgttacaactgtggcattggagttggtcaacttctgctatgacaatgggtccagcgcgtacaccacagtgactggcccatcatcaataccggagatatcactggcacaattggctagtattgtcaaagcttccggcacttcccttagaaaattctgccggtacttcgcgccaataatctggaatctgaggacggacaaaatggctcctgccaattgggaggcttcaggatacaagccaagcgccaaatttgccgcgttcgacttcttcgacggggtggagaatccggcggccatgcaacccccttcgggactaatcaggtcgccgacccaggaagagcggattgccaatgctaccaacaaacaggtgcatctcttccaagccgcggcacaggacaacaactttaccagcaactccgccttcatcaccaaaggccaaatttctgggtcaaccccaaccatccaattccttccaccccccgaataa

SEQ ID NO: 3 - protein, 5 aa, Artificial sequence
  Insertion sequence
TSTTR

SEQ ID NO: 4 - protein, 211 aa, Artificial sequence
  Modified PapMV coat protein CPdeltaN5
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPE

SEQ ID NO: 5 - protein, 222 aa, Artificial sequence
  Modified PapMV coat protein CPsm
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTTRHHHHHH

SEQ ID NO: 6 - DNA, 840 bp, Artificial sequence
  Fusion protein PapMV 3NP-8/183/C (PapMV triple NP)
atggcatccacacccaacatagccaacctgaacgacgcgacctaccagcgtacgcgtgcgctggttcgtaccggtatggacttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaacctgaacgacgcgacctaccagcgtacgcgtgcgctggttcgtaccggtatggacaactttgccagcaactccgccttcatcaccaaaggccaaatttctgggtcaaccccaaccatccaattccttccaccccccgaaactagtaccacgaacctgaacgacgcgacctaccagcgtacgcgtgcgctggttcgtaccggtatggaccgtcaccatcaccatcaccattag

SEQ ID NO: 7 - protein, 22 aa, Artificial sequence
  M2e peptide
SLLTEVETPIRNEWGCRCNDSS

SEQ ID NO: 8 - protein, 23 aa, Artificial sequence
  M2e peptide
SLLTEVETPIRNEWGCRCNDSSD

SEQ ID NO: 9 - protein, 23 aa, Artificial sequence
  M2e peptide
SLLTEVETPIRNEWGCRCNGSSD

SEQ ID NO: 10 - protein, 23 aa, Artificial sequence
  M2e peptide
SLLTEVETPTKNEWDCRCNDSSD

SEQ ID NO: 11 - protein, 23 aa, Artificial sequence
  M2e peptide
SLLTEVETPTRNGWECKCSDSSD

SEQ ID NO: 12 - protein, 23 aa, Artificial sequence
  M2e peptide
SLLTEVETPTRNEWECRCSDSSD

SEQ ID NO: 13 - protein, 8 aa, Artificial sequence
  M2e peptide
EVETPIRN

SEQ ID NO: 14 - protein, 8 aa, Artificial sequence
  M2e peptide
EVETLTRN

SEQ ID NO: 15 - protein, 8 aa, Artificial sequence
  M2e peptide
EVETPIRS

SEQ ID NO: 16 - protein, 8 aa, Artificial sequence
  M2e peptide
EVETPTRN

SEQ ID NO: 17 - protein, 8 aa, Artificial sequence
  M2e peptide
EVETPTKN

SEQ ID NO: 18 - protein, 8 aa, Artificial sequence
  M2e peptide
EVDTLTRN

SEQ ID NO: 19 - protein, 8 aa, Artificial sequence
  M2e peptide
EVETPIRK

SEQ ID NO: 20 - protein, 8 aa, Artificial sequence
  M2e peptide
EVETLTKN

SEQ ID NO: 21 - protein, 9 aa, Artificial sequence
  M2e peptide
EVETPIRNE

SEQ ID NO: 22 - protein, 7 aa, Artificial sequence
  M2e peptide
VETPIRN

SEQ ID NO: 23 - protein, 230 aa, Artificial sequence
  PapMV-M2e fusion construct #1
MAEVETPIRNESTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 24 - protein, 228 aa, Artificial sequence
  PapMV-M2e fusion construct #2
MAVETPIRNSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 25 - protein, 227 aa, Artificial sequence
  PapMV-M2e fusion construct #3
MASEVETPIRNEIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 26 - protein, 225 aa, Artificial sequence
  PapMV-M2e fusion construct #4
MASVETPIRNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 27 - protein, 230 aa, Artificial sequence
  PapMV-M2e fusion construct #5
MASTPNEVETPIRNEIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 28 - protein, 228 aa, Artificial sequence
  PapMV-M2e fusion construct #6
MASTPNVETPIRNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 29 - protein, 16 aa, Artificial sequence
  Salmonella typhi OmpC loop 6 peptide
GTSNGSNPSTSYGFAN

SEQ ID NO: 30 - DNA, 79 bp, Artificial sequence
  Primer
acgtggtacctctaacggttctaacccgtctacctcttacggtttcgcgaacttccccgccatcacccaggaacaaatg

SEQ ID NO: 31 - DNA, 31 bp, Artificial sequence
  Primer
acgtggtaccggctatgttgggtgtggatgc

SEQ ID NO: 32 - DNA, 79 bp, Artificial sequence
  Primer
acgtggtacctctaacggttctaacccgtctacctcttacggtttcgcgaacaacaactttgccagcaactccgccttc

SEQ ID NO: 33 - DNA, 31 bp, Artificial sequence
  Primer
acgtggtaccgtcctgtgccgcggcttggaa

SEQ ID NO: 34 - DNA, 54 bp, Artificial sequence
  Primer
ctagtggtacttctaacggttctaacccgtctacttcttacggtttcgcgaaca

SEQ ID NO: 35 - DNA, 54 bp, Artificial sequence
  Primer
ctagtgttcgcgaaaccgtaagaagtagacgggttagaaccgttagaagtacca

SEQ ID NO: 36 - protein, 9 aa, Artificial sequence
  Influenza NP peptide
TYQRTRALV

SEQ ID NO: 37 - DNA, 59 bp, Artificial sequence
  Primer
agctcgtacgcgtgcgctggttcgtaccggtatggacttccccgccatcacccaggaac

SEQ ID NO: 38 - DNA, 56 bp, Artificial sequence
  Primer
tcgacgtacgctggtaggtcgcgtcgttcaggttggctatgttgggtgtggatgcc

SEQ ID NO: 39 - DNA, 61 bp, Artificial sequence
  Primer
agctcgtacgcgtgcgctggttcgtaccggtatggacaacaactttgccagcaactccgcc

SEQ ID NO: 40 - DNA, 58 bp, Artificial sequence
  Primer
tcgacgtacgctggtaggtcgcgtcgttcaggttgtcctgtgccgcggcttggaagag

SEQ ID NO: 41 - DNA, 58 bp, Artificial sequence
  Primer
acgtcgtacgcgtgcgctggttcgtaccggtatggacacgcgtcaccatcaccatcac

SEQ ID NO: 42 - DNA, 49 bp, Artificial sequence
  Primer
tcgacgtacgctggtaggtcgcgtcgttcaggttactagtttcgggggg

SEQ ID NO: 43 - protein, 5 aa, Artificial sequence
  Flanking sequence
NLNDA

SEQ ID NO: 44 - protein, 5 aa, Artificial sequence
  Flanking sequence
RTGMD

SEQ ID NO: 45 - protein, 12 aa, Artificial sequence
  PapMV coat protein peptide
MASTPNIAFPAI

SEQ ID NO: 46 - protein, 12 aa, artificial sequence
  PapMV coat protein peptide
FPAITQEQMSSI

SEQ ID NO: 47 - protein, 12 aa, Artificial sequence
  PapMV coat protein peptide
MSSIKVDPTSNL

SEQ ID NO: 48 - protein, 12 aa, Artificial sequence
  PapMV coat protein peptide
TSNLLPSQEQLK

SEQ ID NO: 49 - protein, 12 aa, Artificial sequence
  PapMV coat protein peptide
EQLKSVSTLMVA

SEQ ID NO: 50 - protein, 12 aa, Artificial sequence
  PapMV coat protein peptide
LMVAAKVPAASV

SEQ ID NO: 51 - protein, 12 aa, Artificial sequence
  PapMV coat protein peptide
AASVTTVALELV

SEQ ID NO: 52 - protein, 12 aa, Artificial sequence
  PapMV coat protein peptide
LELVNFSYDNGS

SEQ ID NO: 53 - protein, 12 aa, Artificial sequence
  PapMV coat protein peptide
DNGSSAYTTVTG

SEQ ID NO: 54 - protein, 12 aa, Artificial sequence
  PapMV coat protein peptide
TVTGPSSIPEIS

SEQ ID NO: 55 - protein, 12 aa, Artificial sequence
  PapMV coat protein peptide
PEISLAQLASIV

SEQ ID NO: 56 - protein, 12 aa, Artificial sequence
  PapMV coat protein peptide
ASIVKASGTSLR

SEQ ID NO: 57 - protein, 12 aa, Artificial sequence
  PapMV coat protein peptide
TSLRKFSRYFAP

SEQ ID NO: 58 - protein, 12 aa, Artificial sequence
  PapMV coat protein peptide
YFAPIIWNLRTD

SEQ ID NO: 59 - protein, 12 aa, Artificial sequence
  PapMV coat protein peptide
LRTDKMAPANWE

SEQ ID NO: 60 - protein, 12 aa, Artificial sequence
  PapMV coat protein peptide
ANWEASGYKPSA

SEQ ID NO: 61 - protein, 12 aa, Artificial sequence
  PapMV coat protein peptide
KPSAKFAAFDFF

SEQ ID NO: 62 - protein, 12 aa, Artificial sequence
  PapMV coat protein peptide
FDFFDGVENPAA

SEQ ID NO: 63 - protein, 12 aa, Artificial sequence
  PapMV coat protein peptide
NPAAMQPPSGLT

SEQ ID NO: 64 - protein, 12 aa, Artificial sequence
  PapMV coat protein peptide
SGLTRSPTQEER

SEQ ID NO: 65 - protein, 12 aa, Artificial sequence
  PapMV coat protein peptide
QEERIANATNKQ

SEQ ID NO: 66 - protein, 12 aa, Artificial sequence
  PapMV coat protein peptide
TNKQVHLFQAAA

SEQ ID NO: 67 - protein, 12 aa, Artificial sequence
  PapMV coat protein peptide
QAAAQDNNFASN

SEQ ID NO: 68 - protein, 12 aa, Artificial sequence
  PapMV coat protein peptide
FASNSAFITKGQ

SEQ ID NO: 69 - protein, 12 aa, Artificial sequence
  PapMV coat protein peptide
TKGQISGSTPTI

SEQ ID NO: 70 - protein, 11 aa, Artificial sequence
  PapMV coat protein peptide
TPTIQFLPPPE

SEQ ID NO: 71 - protein, 8 aa, Artificial sequence
  PapMV coat protein peptide
PPETSTTR

SEQ ID NO: 72 - DNA, 726 bp, Artificial sequence
  Fusion protein PapMV NP-8
atggcatccacacccaacatagccaacctgaacgacgcgacctaccagcgtacgcgtgcgctggttcgtaccggtatggacttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtaccacgcgtcaccatcaccatcaccattag

SEQ ID NO: 73 - protein, 240 aa, Artificial sequence
  Fusion protein PapMV NP-8
MASTPNIANLNDATYQRTRALVRTGMDFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 74 - DNA, 726 bp, Artificial sequence
  Fusion protein PapMV NP-183
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaacctgaacgacgcgacctaccagcgtacgcgtgcgctggttcgtaccggtatggacaactttgccagcaactccgccttcatcaccaaaggccaaatttctgggtcaaccccaaccatccaattccttccaccccccgaaactagtaccacgcgtcaccatcaccatcaccattag

SEQ ID NO: 75 - protein, 240 aa, Artificial sequence
  Fusion protein PapMV NP-183
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNLNDATYQRTRALVRTGMDNNFASNSAFITKGQISGSTPTIQFLPPPETSTRHHHHHH

SEQ ID NO: 76 - DNA, 726 bp, Artificial sequence
  Fusion protein PapMV NP-C
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaaccaactttgccagcaactccgccttcatcaccaaaggccaaatttctgggtcaaccccaaccatccaattccttccaccccccgaaactagtaccacgaacctgaacgacgcgacctaccagcgtacgcgtgcgctggttcgtaccggtatggacgtcaccatcaccatcaccattag

SEQ ID NO: 77 - protein, 240 aa, Artificial sequence
  Fusion protein PapMV NP-C
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSNLNDATYQRTRALVRTGMDTRHHHHHH

SEQ ID NO: 78 - DNA, 717 bp, Artificial sequence
  Fusion protein PapMV Loop6-8
atggcatccacacccaacatagccggtacctctaacggttctaacccgtctacctcttacggtttcgcgaacttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtaccacgcgtcaccatcaccatcaccattag

SEQ ID NO: 79 - protein, 237 aa, Artificial sequence
  Fusion protein PapMV Loop6-8
MASTPNIAGTSNGSNPSTSYGFANFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 80 - DNA, 717 bp, Artificial sequence
  Fusion protein PapMV Loop6-183
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacggtacctctaacggttctaacccgtctacctcttacggtttcgcgaacaactttgccagcaactccgccttcatcaccaaaggccaaatttctgggtcaaccccaaccatccaattccttccaccccccgaaactagtaccacgcgtcaccatcaccatcaccattag

SEQ ID NO: 81 - protein, 237 aa, Artificial sequence
  Fusion protein PapMV Loop6-183
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDGTSNGSNPSTSYGFANNNFASNSAFITKGQISGSTPTIQFLPPPETSTRHHHHHH

SEQ ID NO: 82 - DNA, 718 bp, Artificial sequence
  Fusion protein PapMV Loop6-C
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaaccaactttgccagcaactccgccttcatcaccaaaggccaaatttctgggtcaaccccaaccatccaattccttccaccccccgaaactagtaccacgggtacctctaacggttctaacccgtctacctcttacggtttcgcgaaccgtcaccatcaccatcaccattag

SEQ ID NO: 83 - protein, 240 aa, Artificial sequence; identical to SEQ ID NO: 77
  Fusion protein PapMV Loop6-C

SEQ ID NO: 84 - DNA, 751 bp, Artificial sequence
  Fusion protein PapMV 3NP-C
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaaccaactttgccagcaactccgccttcatcaccaaaggccaaatttctgggtcaaccccaaccatccaattccttccaccccccgaaactagtaccacgacctaccagcgtacgcgtgcgctggttacctaccagcgtacgcgtgcgctggttacctaccagcgtacgcgtgcgctggttcgtcaccatcaccatcaccattag

SEQ ID NO: 85 - protein, 248 aa, Artificial sequence
  Fusion protein PapMV 3NP-C
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTYQRTRALVTYQRTRALVTYQRTRALVTRHHHHHH

SEQ ID NO: 86 - DNA, 783 bp, Artificial sequence
  Fusion protein PapMV NP-8/183
atggcatccacacccaacatagccaacctgaacgacgcgacctaccagcgtacgcgtgcgctggttcgtaccggtatggacttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaacctgaacgacgcgacctaccagcgtacgcgtgcgctggttcgtaccggtatggacaactttgccagcaactccgccttcatcaccaaaggccaaatttctgggtcaaccccaaccatccaattccttccaccccccgaaactagtaccacgcgtcaccatcaccatcaccattag

SEQ ID NO: 87 - protein, 259 aa, Artificial sequence
  Fusion protein PapMV NP-8/183
MASTPNIANLNDATYQRTRALVRTGMDFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNLNDATYQRTRALVRTGMDNNFASNSAFITKGQISGSTPTIQFLPPPETSTRHHHHHH

SEQ ID NO: 88 - DNA, 783 bp, Artificial sequence
  Fusion protein PapMV NP-8/C
atggcatccacacccaacatagccaacctgaacgacgcgacctaccagcgtacgcgtgcgctggttcgtaccggtatggacttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtaccacgaacctgaacgacgcgacctaccagcgtacgcgtgcgctggttcgtaccggtatggaccgtcaccatcaccatcaccattag

SEQ ID NO: 89 - protein, 259 aa, Artificial sequence
  Fusion protein PapMV NP-8/C
MASTPNIANLNDATYQRTRALVRTGMDFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSNLNDATYQRTRALVRTGMDTRHHHHHH

SEQ ID NO: 90 - DNA, 783 bp, Artificial sequence
  Fusion protein PapMV NP-183/C
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaacctgaacgacgcgacctaccagcgtacgcgtgcgctggttcgtaccggtatggacaactttgccagcaactccgccttcatcaccaaaggccaaatttctgggtcaaccccaaccatccaattccttccaccccccgaaactagtaccacgaacctgaacgacgcgacctaccagcgtacgcgtgcgctggttcgtaccggtatggaccgtcaccatcaccatcaccattag

SEQ ID NO: 91 - protein, 259 aa, Artificial sequence
  Fusion protein PapMV NP-183/C
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNLNDATYQRTRALVRTGMDNNFASNSAFITKGQISGSTPTIQFLPPPETSNLNDATYQRTRALVRTGMDTRHHHHHH

SEQ ID NO: 92 - DNA, 751 bp, Artificial sequence
  Fusion protein PapMV 3NP-8
atggcatccacacccaacatagccacctaccagcgtacgcgtgcgctggttacctaccagcgtacgcgtgcgctggttacctaccagcgtacgcgtgcgctggttttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaaccaactttgccagcaactccgccttcatcaccaaaggccaaatttctgggtcaaccccaaccatccaattccttccaccccccgaaactagtaccacgcgtcaccatcaccatcaccattag

SEQ ID NO: 93 - protein, 248 aa, Artificial sequence
  Fusion protein PapMV 3NP-8
MASTPNIATYQRTRALVTYQRTRALVTYQRTRALVFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTRHHHHHH

SEQ ID NO: 94 - protein, 278 aa, Artificial sequence
  Fusion protein PapMV 3NP-8/183/C (PapMV triple NP)
MASTPNIANLNDATYQRTRALVRTGMDFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNLNDATYQRTRALVRTGMDNNFASNSAFITKGQISGSTPTIQFLPPPETSNLNDATYQRTRALVRTGMDTRHHHHHH

SEQ ID NO: 95 - protein, 8 aa, Artificial sequence
  M2e derived peptide
EVXTXXXX

SEQ ID NO: 96 - protein, 7 aa, Artificial sequence
  M2e derived peptide
VXTXXXX

SEQ ID NO: 97 - protein, 7 aa, Artificial sequence
  M2e derived peptide
VETPIRN

SEQ ID NO: 98 - protein, 7 aa, Artificial sequence
  M2e derived peptide
VETLTRN

SEQ ID NO: 99 - protein, 7 aa, Artificial sequence
  M2e derived peptide
VETPIRS

SEQ ID NO: 100 - protein, 7 aa, Artificial sequence
  M2e derived peptide
VETPTRN

SEQ ID NO: 101 - protein, 7 aa, Artificial sequence
  M2e derived peptide
VETPTKN

SEQ ID NO: 102 - protein, 7 aa, Artificial sequence
  M2e derived peptide
VDTLTRN

SEQ ID NO: 103 - protein, 7 aa, Artificial sequence
  M2e derived peptide
VETPIRK

SEQ ID NO: 104 - protein, 7 aa, Artificial sequence
  M2e derived peptide
VETLTKN